(19) United States
(12) Patent Application Publication
(10) Pub. No.: US 2025/0303040 A1
(43) Pub. Date: Oct. 2, 2025

(54) SYSTEM AND METHOD OF TREATMENT FOR PREMATURE FETUS

(71) Applicants: THE CHILDREN'S HOSPITAL OF PHILADELPHIA, Philadelphia, PA (US); VITARA BIOMEDICAL, INC., Philadelphia, PA (US)

(72) Inventors: James S. TARMIN, Penn Valley, PA (US); Monica LOCKARD, Elkins Park, PA (US); Christopher C. GREGORY, Newtown, PA (US); Maria RENZI, Philadelphia, PA (US); Marcus Graeme DAVEY, Ardmore, PA (US)

(21) Appl. No.: 18/863,266

(22) PCT Filed: May 8, 2023
(86) PCT No.: PCT/US2023/066745

§ 371 Date: Nov. 5, 2024

Related U.S. Application Data
(60) Provisional application No. 63/364,313, filed on May 6, 2022.

(51) Int. Cl. A61M 1/16; A61G 11/00; A61G 13/00; A61N 5/06

(52) U.S. Cl. CPC A61M 1/1698; A61G 11/007; A61G 11/009; A61G 13/009; A61N 5/0621; A61M 2205/3351; A61M 2205/3355; A61M 2240/00

(57) ABSTRACT

A system configured to enclose a premature fetus within an extracorporeal environment to promote growth of the fetus and increase viability of the fetus, The system includes a chamber having an interior space configured to enclose the fetus, a first fluid circuit that delivers sterile fluid to the chamber, and a second fluid system that transfers oxygen to the fetus. The system chamber includes a stop mechanism including a clamp and an actuator, the clanip positioned in the interior space, the actuator coupled to the clamp such that movement of the actuator moves the clamp, and the actuator positioned at least partially outside the interior space. A method of using the system to treat hyperbilirubinemia is also provided.

### CROSS-REFERENCE TO RELATED APPLICATIONS

This application claims the benefit of U.S. Provisional Application Ser. No. 63/364,313 filed May 6, 2022, the contents of which is hereby incorporated by reference as if set forth in its entirety herein.

### TECHNICAL FIELD

The present disclosure relates generally to neonatal care. More specifically, the present disclosure describes devices, systems, and methods related to improving the viability of a premature fetus outside of the womb. According to one aspect, the present disclosure relates to improving viability of premature fetuses at a stage of development prior to 28 weeks gestation.

### BACKGROUND

Extreme prematurity is the leading cause of infant morbidity and mortality in the United States, with over one third of all infant deaths and one half of cerebral palsy diagnoses attributed to prematurity. The 2010 Center for Disease Control National Vital Statistics Report notes birth rates at a gestational age of less than 28 weeks in the United States over roughly the past decade have remained stable at approximately 0.7%, or 30,000 births annually. Similarly, birth rates at gestational ages 28-32 weeks over the past decade in the United States have been stable at 1.2%, or 50,000 births annually.

Premature birth may occur due to any one of a multitude of reasons. For example, premature birth may occur spontaneously due to preterm rupture of the membranes (PROM), structural uterine features such as shortened cervix, secondary to traumatic or infectious stimuli, or due to multiple gestation. Preterm labor and delivery is also frequently encountered in the context of fetoscopy or fetal surgery, where instrumentation of the uterus often stimulates uncontrolled labor despite maximal tocolytic therapy.

Respiratory failure represents the most common and challenging problem associated with extreme prematurity, as gas exchange in critically preterm neonates is impaired by structural and functional immaturity of the lungs. Advances in neonatal intensive care have achieved improved survival and pushed the limits of viability of preterm neonates to 22 to 24 weeks gestation, which marks the transition from the canalicular to the saccular phase of lung development. Although survival has become possible, there is still a high rate of chronic lung disease and other complications of organ immaturity, particularly in fetuses born prior to 28 weeks gestation. The development of a system that could support normal fetal growth and organ maturation for even a few weeks could significantly reduce the morbidity and mortality of extreme prematurity and improve quality of life in survivors.

The development of an “artificial placenta” has been the subject of investigation for over 50 years with little success. Previous attempts to achieve adequate oxygenation of the fetus in animal models have employed traditional extracorporeal membrane oxygenation (ECMO) with pump support and have been limited by circulatory overload and cardiac failure in treated animals. The known systems have suffered from unacceptable complications, including: 1) progressive circulatory failure due to after-load or pre-load imbalance imposed on the fetal heart by oxygenator resistance or by circuits incorporating various pumps; and 2) contamination and fetal sepsis.

In fetal patients, bilirubin is normally produced during the breakdown of red blood cells, the rate of which is increased during periods of rapid somatic growth (i.e., during fetal life). Normally, fetal serum bilirubin is managed by the placenta in utero. However, extreme premature babies, such as those in the standard of care (“SOC”) incubators or in artificial placenta systems, cannot have their serum bilirubin level managed by the placenta. SOC patients in incubators undergo phototherapy using a bilirubin phototherapy device. These phototherapy devices expose the baby in the incubator to a specific wavelength of light which chemically changes the blood bilirubin via photoisomerization to a more manageable form that is excreted through the baby's urine. Babies that undergo phototherapy treatment in incubators also require eye protection to prevent damage from the light rays.

Accordingly, a system and method configured to provide extracorporeal support for a premature fetus, or fetuses (preterm or term) with inadequate respiratory gas exchange to support life, due to a spectrum of conditions/disorders, may improve viability.

### SUMMARY

According to one aspect of the disclosure, described herein is a system and method to treat a premature fetus in an extracorporeal system. As used herein, the term “treating” includes lowering serum bilirubin levels, halting the increase of serum bilirubin levels, or both. After treating portion of the blood in the oxygenation circuit, a clinical target of about 2 to about 4 milligrams/deciliter (mg/dL) per day total bilirubin (tBili) reduction can be achieved using the method and device described herein.

According to another aspect of the disclosure, a method is provided for treating a level of bilirubinemia in a blood of a premature fetus, the method comprising the steps of a) connecting a premature fetus to an ex-utero system as disclosed herein and configured to provide oxygen to the fetus, wherein the connecting step comprises the steps of attaching a first cannula to a vein of the umbilical cord, attaching a second cannula to a first artery of the umbilical cord, attaching a third cannula to a second artery of the umbilical cord, and connecting one or more of the first, second and third cannulae to the oxygenator via an oxygenation circuit, wherein at least one of the cannulae is in fluid connection with the fetus' circulatory system and the umbilical cord is connected to the oxygenator such that deoxygenated blood is delivered from the fetus to the oxygenator, and oxygenated blood is delivered from the oxygenator to the fetus, and wherein the system further comprises a port in fluid connection with the fetus' circulatory system; and b) exposing at least a portion of the oxygenation circuit to a light source comprising a light output ranging from about 430 to about 490 nanometers (nm), or from about 460 nm to about 490 nm, or from about 470 to about 490 nm.

### BRIEF DESCRIPTION OF THE DRAWINGS

The foregoing summary, as well as the following detailed description of illustrative embodiments of the application, will be better understood when read in conjunction with the appended drawings. For the purposes of illustrating the present disclosure, there is shown in the drawings illustrative embodiments. It should be understood, however, that the application is not limited to the specific embodiments and methods disclosed, and reference is made to the claims for that purpose. In the drawings:

FIG. 1 is a first isometric view of an extracorporeal support system according to one embodiment, the extracorporeal support system in a first configuration;

FIG. 2 is a second isometric view of the extracorporeal support system illustrated in FIG. 1, the extracorporeal support system in the first configuration;

FIG. 3 is a third isometric view of a portion of the extracorporeal support system illustrated in FIG. 1, the extracorporeal support system in the first configuration;

FIG. 4 is an isometric view of the extracorporeal support system illustrated in FIG. 1, the extracorporeal support system in a second configuration;

FIG. 5 is an isometric view of a fetal chamber of the extracorporeal support system according to one embodiment, the fetal chamber in a first configuration;

FIG. 6 is a top plan view of the fetal chamber illustrated in FIG. 5, the fetal chamber in a second configuration;

FIG. 7 is a top plan view of a first member of a volume adjustment assembly;

FIG. 8 is a top plan view of a second member of a volume adjustment assembly;

FIG. 9 is a top plan view of a third member of a volume adjustment assembly;

FIG. 10 is a schematic view of the extracorporeal support system illustrated in FIG. 1, in use in an operating room;

FIG. 11 is an isometric view of a portion of the fetal chamber illustrated in FIG. 5, the portion including an emergency clamp assembly, the emergency clamp assembly is a first configuration;

FIG. 12 is an isometric view of a portion of the fetal chamber illustrated in FIG. 5, the portion including the emergency clamp assembly in a second configuration;

FIG. 13 is an isometric view of a seal of the fetal chamber according to one aspect of the disclosure;

FIG. 14 is a cross-sectional view of the fetal chamber illustrated in FIG. 5 along line 14-14, the fetal chamber including a port;

FIG. 15 is an isometric view of the port illustrated in FIG. 14, according to one embodiment, in a first configuration;

FIG. 16 is a cross-sectional view of the port illustrated in FIG. 15 in the first configuration;

FIG. 17 is a cross-sectional view of the port illustrated in FIG. 15 in a second configuration;

FIG. 18 is a cross-sectional view of the port illustrated in FIG. 15 in the second configuration, and a suction device;

FIG. 19 is a cross-sectional view of a port of the extra corporeal support system, according to another embodiment;

FIG. 20 is a cross-sectional view of the port illustrated in FIG. 19, and a suction device, the suction device in a first position;

FIG. 21 is a cross-sectional view of the port and suction device illustrated in FIG. 20, the section device in a second position;

FIG. 22 is a cross-sectional view of the port and suction device illustrated in FIG. 20, the section device in a third position;

FIG. 23 is an isometric view of a port of the extra corporeal support system, according to another embodiment;

FIG. 24 is a side cross-sectional view of the port illustrated in FIG. 23 and a suction device in a first position relative to the port;

FIG. 25 is a side cross-sectional view of the port and the suction device illustrated in FIG. 25, the suction device in a second position relative to the port;

FIG. 26 is a side cross-sectional view of the port and the suction device illustrated in FIG. 25, the suction device in a third position relative to the port;

FIG. 27 is a schematic view of a first fluid circuit of the extracorporeal support system illustrated in FIG. 1;

FIG. 28 is an isometric view of a pressure regulator of the first fluid circuit illustrated in FIG. 27;

FIG. 29 is an isometric view of a sterilization unit of the extracorporeal support system illustrated in FIG. 1, the sterilization unit in a first configuration;

FIG. 30 is an isometric view of the sterilization unit illustrated in FIG. 29, the sterilization unit in a second configuration;

FIG. 31 is a schematic view of a second fluid circuit of the extracorporeal support system illustrated in FIG. 1;

FIG. 32 is a schematic view of an embodiment of a photo-therapy device suitable for use in a method disclosed herein;

FIG. 33 is a schematic view of a second embodiment of a photo-therapy device suitable for use in a method disclosed herein;

FIG. 34 is a schematic view of a third embodiment of a photo-therapy device suitable for use in a method disclosed herein;

FIG. 35 is a schematic view of a fourth embodiment of a photo-therapy device suitable for use in a method disclosed herein;

FIG. 36 is an isometric view of an embodiment of the oxygenator in the extracorporeal system having a face plate, wherein at least a portion of the face plate is transparent;

FIG. 37 shows the relationship between total bilirubin within a blood sample and time, wherein the blood sample has been treated with an embodiment of the present invention;

FIG. 38 shows the relationship between direct bilirubin within a blood sample and time, wherein the blood sample has been treated with an embodiment of the present invention;

FIG. 39 shows an isometric view of an embodiment of the oxygenator in the extracorporeal system with a light assembly adjacent the oxygenator to expose blood within the oxygenator to light from a phototherapy device;

FIG. 40 is an isometric view of an embodiment of a canopy that receives the chamber and the phototherapy device; and

FIG. 41 is a bottom isometric view of the canopy of FIG. 40.

### DETAILED DESCRIPTION OF ILLUSTRATIVE EMBODIMENTS

Aspects of the disclosure will now be described in detail with reference to the drawings, wherein like reference numbers refer to like elements throughout, unless specified otherwise. Certain terminology is used in the following description for convenience only and is not limiting. The term “plurality”, as used herein, means more than one. The terms “a portion” and “at least a portion” of a structure include the entirety of the structure. Certain features of the disclosure which are described herein in the context of separate embodiments may also be provided in combination in a single embodiment. Conversely, various features of the disclosure that are described in the context of a single embodiment may also be provided separately or in any subcombination.

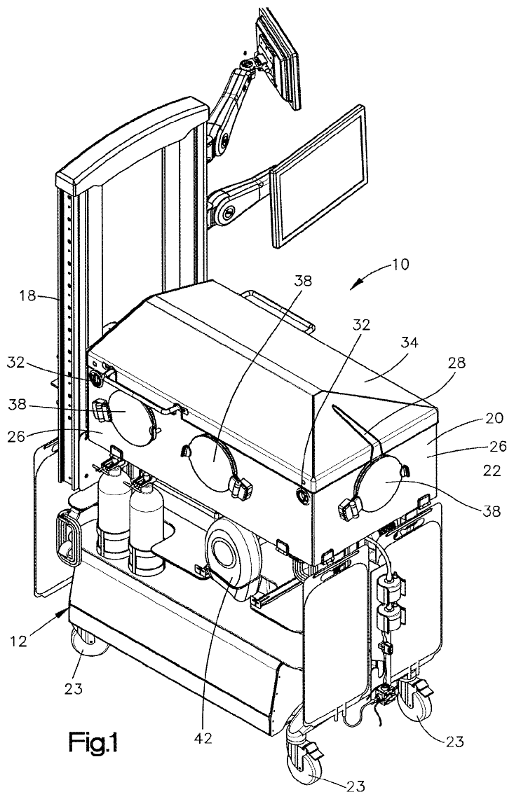

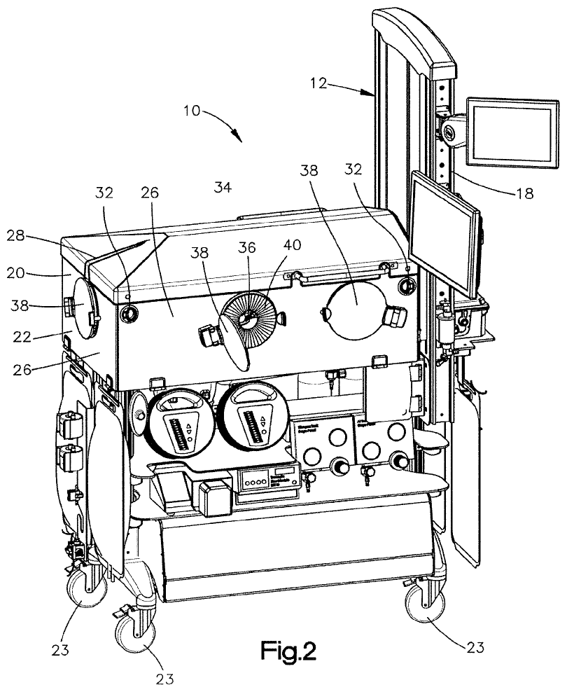

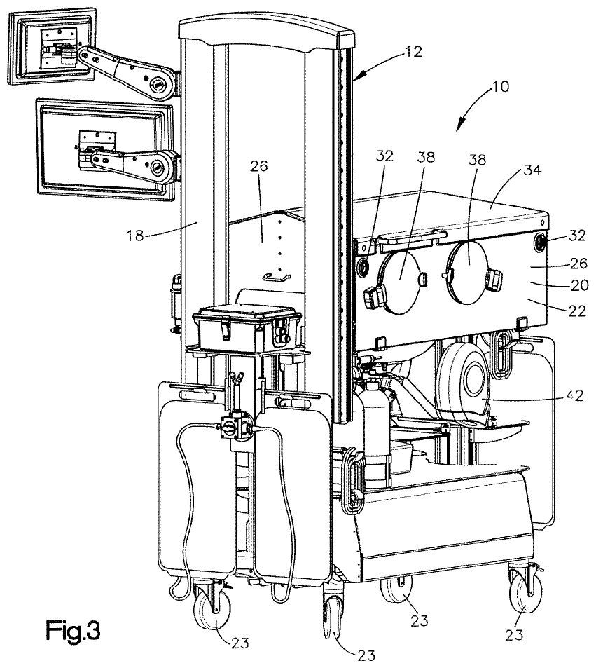

Referring to FIGS. 1 to 4, a system 10 is configured to provide extracorporeal support to a premature fetus. According to one aspect of the disclosure the system 10 is configured to provide a system environment that is similar to an environment the premature fetus would experience in utero. Viability of a premature fetus that is removed from the uterine environment and that is, for example, between about 22 weeks to about 24 weeks gestation, may be increased by placing the premature fetus in the system environment. According to one aspect of the disclosure, the system environment is configured to: 1) limit exposure of the premature fetus to light; 2) limit exposure of the premature fetus to sound; 3) maintain the fetus submerged within a liquid environment; 4) maintain the premature fetus within a desired temperature range; or 5) any combination thereof. Some non-limiting examples of extracorporeal systems suitable for the treatment of the premature fetus described herein are found in the following: US Publ. No. 2021/0052453 entitled “Extracorporeal Life Support System and Methods of Use Thereof”; US Publ. No. 2021/0161744 entitled “Method And Apparatus For Extracorporeal Support Of Premature Infants”; and US Publ. No. 2023/0000706 entitled “System and Method Configured to Provide Extracorporeal Support for Premature Fetus” which are incorporated herein by reference in their entirety.

The system 10 includes a cart 12 having a frame 18 and a housing 20. The frame 18 is configured to support the housing 20 such that the housing 20 is configured to at least partially contribute to providing the system environment to the premature fetus. The housing 20 includes one or more housing members 22 that at least partially define an interior space 24 that contains the system environment. As shown in the illustrated embodiment, the housing members 22 may include a plurality of side walls 26, a lid 28, and a base 30. According to one aspect of the disclosure, at least one of the plurality of side walls 26, the lid 28, or both are moveable.

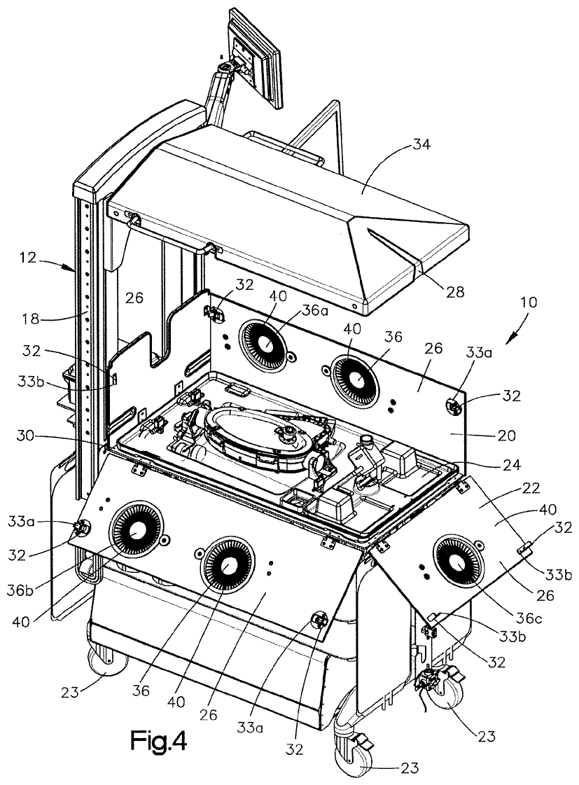

The housing 20 defines a first configuration, an example of which is shown in FIGS. 1, 2, and 3, in which the plurality of side walls 26 and the lid 28 are arranged to cooperatively define the interior space 24. In the first configuration the housing 20 is configured to maintain the system environment and restrict access to the interior space 24. The housing 20 defines a second configuration, an example of which is shown in FIG. 4, in which the plurality of side walls 26 and the lid 28 are arranged to provide increased access to the interior space 24.

As shown in the illustrated embodiment, the housing 20 may include four side walls 26. One or more of the side walls 26, for example all four, three, two, or one of the side walls 26, may be pivotally coupled to the frame 18. As shown in FIGS. 1, 2, and 3, in the first configuration the side walls 26, the lid 28, and the base 30 cooperate to define the interior space 24. As shown in FIG. 4, in the second configuration one or more of the side walls 26 are pivoted away from others of the side walls 26 such that the interior space 24 is accessible from an exterior of the system 10. The housing 20 may include a locking mechanism 32 configured to: 1) secure the side walls 26 in the first configuration when the locking mechanism 32 is engaged, and 2) allow the side walls 26 to pivot when the locking mechanism 32 is disengaged.

As shown in the illustrated embodiment, the locking mechanism 32 may include corresponding members located on adjacent ones of the plurality of side walls 26. For example the locking mechanism may include a latch 33a located on one of the side walls 26 and a projection 33b configured to be captured by the latch 33a thereby securing the adjacent ones of the plurality of side walls 26 to one another.

As shown in the illustrated embodiment, the lid 28 may be translatable with respect to the side walls 26. The system 10 may be configured such that the lid 28 is translatable along a vertical direction, which is substantially perpendicular to a surface upon which the system 10 is positioned, for example a floor, such as a hospital floor. According to one aspect of the disclosure, in the first configuration the lid 28 is in close proximity, for example touching, one or more of the side walls 26, and in the second configuration an entirety of the lid 28 is spaced from the floor a distance of at least about six feet, for example about seventy-one inches. The system 10 may be further configured such that when the entirety of the lid 28 is spaced from the floor by a distance of at least about six feet, an entirety of the system 10 is positioned less than seventy-nine inches from the floor.

The amount of clearance under the lid 28 is configured to allow a maximum amount of space for a person, such as a doctor or nurse, to access the system 10 without interference from the lid 28, and the maximum height of the system 10 is configured to allow the system 10 to pass through a standard size hospital doorway. According to another aspect of the disclosure, in the second configuration an entirety of the lid 28 may be spaced from the floor a distance less than six feet, a portion of the system 10 may be spaced from the floor by a distance greater than seventy-nine inches, or both.

According to another embodiment, the side walls 26 and the lid 28 may be an integral or monolithic piece. According to another embodiment, the side walls 26 and the lid 28, whether separate or monolithic, may be translatable, pivotable, or both relative to the frame 18.

The lid 28 may be transparent such that a person outside of the system 10 can view the interior space 24 of the system 10 when the housing 20 is in the first configuration. The housing 20 may further include a removable, opaque cover 34 that prevents light from a source outside of the interior space 24 from reaching the interior space 24 through the transparent lid 28. The system 10 may be configured to limit the amount of light that reaches the interior space 24 when the system 10 is in the first configuration to about 1.2 lux or below. According to one embodiment, the opaque cover 34 may be secured to the housing 20 magnetically. Alternatively, the lid 28 may be opaque. The system 10 may be configured to provide an indirect view of the interior space 24, for example the system 10 may include a camera positioned inside the interior space 24 that transmits an image to a screen outside the interior space 24.

The housing 20 may be configured such that the lid 28 remains in the second configuration without a person exerting an external force on the lid 28. For example, the housing 20 may include a constant force spring assembly that provides a retention force that holds the lid 28 at its current distance from the floor until an external force, in addition to the force of gravity, is applied to the lid 28.

The housing 20 may be configured to provide access to the interior space 24 when the housing 20 is in the first configuration. As shown in the illustrated embodiment, the housing 20 includes one or more access ports 36 that are each configured to provide a passageway for a person's hand from an exterior of the system 10 to the interior space 24. The access ports 36 may each include a respective cover 38 configured to block the access port 36. Each of the respective covers 38 may be configured to be moved relative to the access port 36 thereby providing access to the passageway. The access port 36 may include a flexible iris 40 configured to provide a seal around an arm of the person using the access port 36 to access the interior space 24.

According to one aspect of the disclosure, the access ports 36 are positioned such that a portion of the access port 36 is between about 3 feet and about 4 feet from the floor. For example, a center of the access port 36 may be positioned about forty-three inches from the floor. The access ports 36 may be positioned such that different ones of the access ports 36 provide access to the interior space 24 along different directions. As shown in the illustrated embodiment, the system 10 may include: a first one of the access ports 36a configured to provide access to the interior space 24 along a first direction; a second one of the access ports 36b configured to provide access to the interior space 24 along a second direction that is opposite the first direction; a third one of the access ports 36c configured to provide access to the interior space 24 along a third direction that is perpendicular to both the first direction and the second direction, or any combination thereof.

The system 10, according to one embodiment, may include a heater 42 configured to maintain the interior space 24 within a desired temperature range. For example, the heater 42 may be configured to maintain a temperature within the interior space 24 between about twenty-eight degrees Celsius and about thirty-eight degrees Celsius, preferably between about thirty degrees Celsius and about thirty-four degrees Celsius, for example about thirty-two degrees Celsius. The system 10 according to one embodiment, may include a sound dampening system configured to dampen the amplitude of sound that reaches the interior space 24 of the system 10 when the housing is in the first configuration to below about 20 decibels. According to one embodiment, the sound dampening can be achieved by attaching sound dampening material to one or more of the side walls 26.

The system 10 may be mobile such that system is configured to be moveable from one location to another while still providing appropriate levels of oxygen delivery to the interior space 24, for example to a fetus in the interior space 24. According to one aspect of the disclosure, the system 10 may include a plurality of wheels 23 enabling the system 10 to be moved, for example from an operating room to a neonatal intensive care unit. As shown in the illustrated embodiment, the wheels 23 are coupled to the cart 12.

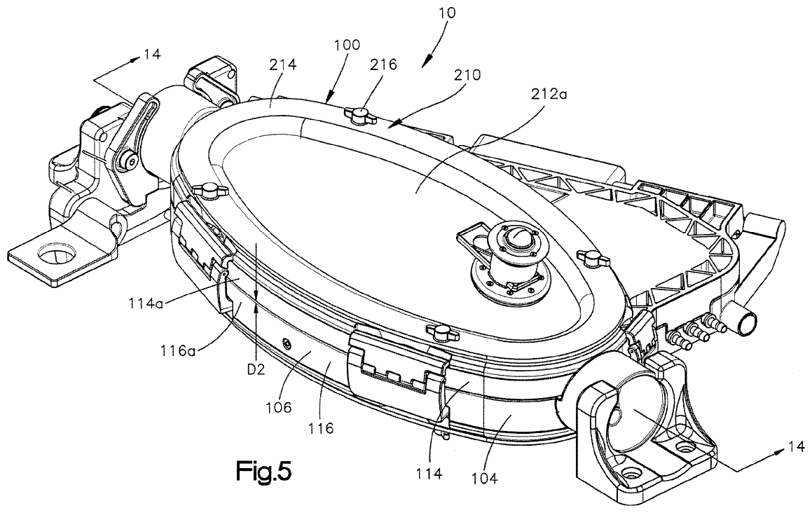

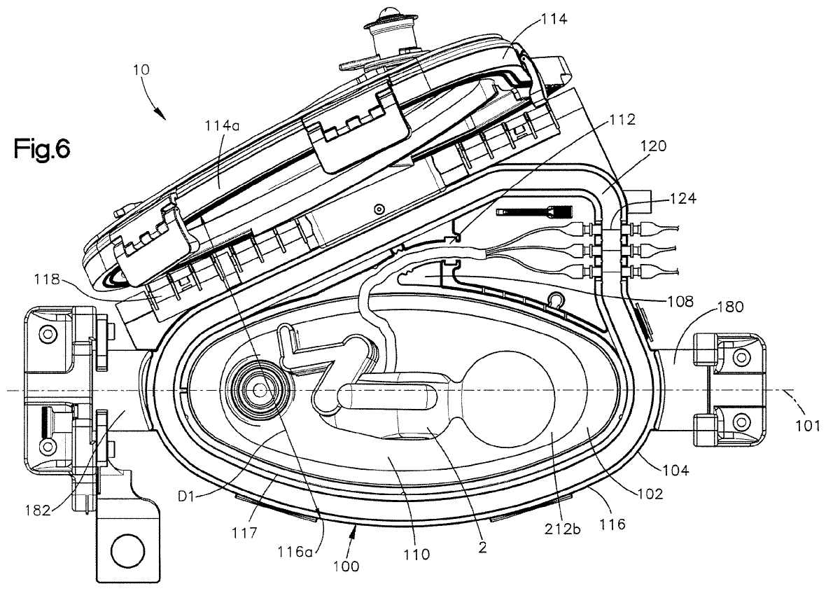

Referring to FIGS. 5 and 6, the system 10 includes a chamber 100 configured to receive and enclose a fetus 2, for example a premature, human fetus, within an extra uterine, enclosed environment. According to one aspect of the disclosure, the cart 12 is configured to enclose the chamber 100 within the interior space 24. The chamber 100 defines a chamber interior space 102 that contains the extra uterine, enclosed environment. The chamber 100 defines a first configuration, also referred to herein as an open configuration, as shown in FIG. 6. In the open configuration the chamber interior space 102 is accessible such that the chamber interior space 102 is configured to receive the fetus 2. The chamber 100 defines a second configuration, also referred to herein as a closed configuration, as shown in FIG. 5. In the closed configuration the chamber interior space 102 is sealed off from the environment surrounding the chamber 100.

The chamber 100 includes a chamber housing 104 that defines the chamber interior space 102. According to one aspect of the disclosure, the chamber housing 104 may include an outer chamber wall 106 that defines an outer boundary of the chamber interior space 102. As shown in the illustrated embodiment, the outer chamber wall 106 defines an outer perimeter of the chamber 100 when the chamber 100 is in the closed configuration. The chamber housing 104 may further include an inner chamber wall 108 that at least partially separates a first portion 110 of the chamber interior space 102 from a second portion 112 of the chamber interior space 102.

According to one aspect of the disclosure, when the chamber 100 is in the open configuration a first shell 114 of the housing and a second shell 116 of the housing cooperatively define an opening 117 into the interior chamber space 102, the opening 117 defines a first distance D1 measured from a portion 114a of the first shell 114 to a portion 116a of the second shell 116. When the chamber 100 is in the closed configuration the opening 117 defines a second distance D2 measured from the portion 114a of the first shell 114 to the portion 116a of the second shell 116. The second distance D2 is less than the first distance D1. As shown in the illustrated embodiment, the second distance D2 may be zero such that the portion 114a and the portion 116a abut. According to another embodiment D2 may be greater than zero.

The system 10 may be configured such that the chamber 100 is rotatable about an axis 101, relative to the cart 12. The axis 101 may be a longitudinal axis that the chamber 100 is elongate along. As shown in the illustrated embodiment, the axis 101 may pass through a first end 180 of the chamber 100 and a second end 182 of the chamber 100. According to one embodiment, the chamber 100 is rotatable about the axis 101 through a full revolution of 360 degrees. According to another embodiment, the chamber 100 is rotatable about the axis 101 through less than a full revolution of 360 degrees. For example, the chamber 100 may be rotatable about the axis 101 about 180 degrees clockwise from the position shown in FIG. 6, and rotatable about 180 degrees counterclockwise from the position shown in FIG. 6. The system 10 being configured such that the chamber 100 is rotatable about the axis 101 relative to the cart 12 allows the position of the fetus 2 to be adjusted while maintaining the chamber 100 in the closed configuration. Adjustment of the position of the fetus 2 may reduce or eliminate dependent edema, fetal asymmetry, pressure sores, or other undesired conditions.

The chamber 100 defines a length measured along the axis 101, a width measured along a lateral direction perpendicular to the axis 101, and a height measured along a vertical direction that is perpendicular to both the axis 101 and the lateral direction. According to one embodiment, the chamber 100 defines a maximum length measured along the axis 101, a maximum width measured along a line that is perpendicular to the axis 101 and that intersects the first shell 114 in two separate locations, and a maximum height measured along a line that is perpendicular to both the axis 101 and the lateral direction and that intersects both the first shell 114 and the second shell 116 when the chamber 100 is in the closed configuration. As shown in the illustrated embodiment, the chamber 100 may be configured such that the maximum length is greater than the maximum width, and the maximum width is greater than the maximum height.

The chamber 100 may include a volume adjustment assembly 210 configured to change, for example increase, decrease, or both, a volume defined by the interior space 102 when the chamber 100 is in the closed configuration. The chamber 100 may include at least one flexible wall 212. As shown in the illustrated embodiment, the first shell 114 may include a first flexible wall 212a and the second shell 116 may include a second flexible wall 212b. The volume adjustment assembly 210 may include a member 214 configured to be coupled to the chamber housing 104 such that the member 214 deforms at least one of the flexible walls 212 thereby reducing the volume of the interior space 102 compared to when the member 214 is not coupled to the chamber housing 104.

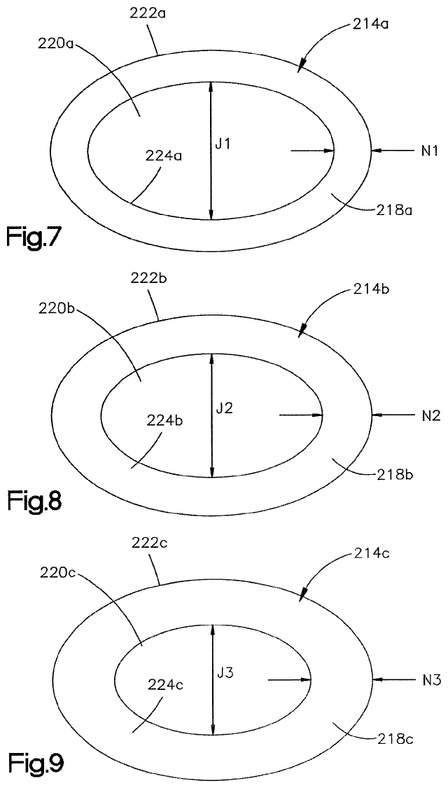

Referring to FIGS. 7 to 9, according to one aspect of the disclosure, the volume adjustment assembly 210 includes a plurality of members 214. Each of the plurality of member 214 may be configured to limit the maximum value of the volume of the interior space 102 when the chamber 100 is in the closed configuration. For example, the plurality of members 214 may each define an outer ring 218 and an opening 220 defined by the outer ring 218 such that the opening 220 is at least partially enclosed by the outer ring 218 within a plane P1. The plurality of members 214 may include a first member 214a, a second member 214b, a third member 214c.

As shown in the illustrated embodiment, the opening 220a of the first member 214a defines a first cross-sectional area J1 measured within the plane P1. The opening 220b of the second member 214b defines a second cross-sectional area J2 measured within the plane P1. The opening 220c of the third member 214c defines a third cross-sectional area J3 measured within the plane P1. According to one aspect of the disclosure, the plurality of members 214 are configured such that the first cross-area J1 is greater than the second cross-sectional area J2, and the second cross-sectional area J2 is greater than the third cross-sectional area J3. The plurality of members 214 may further be configured such that the third member 214c restricts the maximum volume of the interior space 102 more than when the third member 214c is coupled to the chamber housing 104 than when the second member 214b is coupled to the chamber housing 104. The plurality of members 214 may further be configured such that the second member 214b restricts the maximum volume of the interior space 102 when the second member 214b is coupled to the chamber housing 104 more than when the first member 214a is coupled to the chamber housing 104.

According to one aspect of the disclosure, the maximum volume of the interior space 102 without any of the plurality of members 214 attached may be about 3.6 Liters. The plurality of members 2144 may be configured such that the third member 214c restricts the maximum volume of the interior space 102 to a volume sufficient to accommodate a fetus of about 22 weeks estimated gestational age, the second member 214b restricts the maximum volume of the interior space 102 to a volume sufficient to accommodate a fetus of about 24 weeks estimated gestational age, the first member 214a restricts the maximum volume of the interior space 102 to a volume sufficient to accommodate a fetus of about 26 weeks estimated gestational age, or any combination thereof.

According to one aspect of the disclosure the plurality of members 2144 may be configured such that the third member 214c restricts the maximum volume of the interior space 102 to a volume of about 15 liters, the second member 214b restricts the maximum volume of the interior space 102 to a volume of about 2 liters, the first member 214a restricts the maximum volume of the interior space 102 to a volume of about 2.5 liters, or any combination thereof.

According to one aspect of the disclosure, the ring 218a of the first member 214a, the second member 214b, and the third member 214c each define an equal outer perimeter 222a, 222b, and 222c, and different inner perimeters 224a, 224b, and 224c, respectively. Thus the first member 214a, the second member 214b, and the third member 214c may each define different thickness N1, N2, and N3, respectively, measured in the plane P1.

Thus the plurality of members 214 may be configured to be sequentially coupled to the chamber housing 104 to incrementally increase the maximum volume of the interior space 102 to correspond to growth of the fetus 2. For example, the chamber 100 may be configured for use such that when the fetus 2 is first placed inside the interior space 102, the third member 214c is coupled to the chamber housing 104 to restrict the maximum volume of the interior space 102 and limit movement, for example rotation, of the fetus 2 within the interior space 2. As the fetus 2 develops and grows, more volume within the interior space 102 may be needed. Accordingly, the third member 214c may be decoupled from the chamber housing 104 and the second member 214b may be coupled to the chamber housing 104, all while the chamber 100 remains in the closed configuration. With the second member 214b coupled to the chamber housing 104, the maximum volume of the interior space 102 is greater than it was within the third member 214c coupled to the chamber housing 104.

As the fetus 2 develops and grows further, more volume within the interior space 102 may be needed. Accordingly, the second member 214b may be decoupled from the chamber housing 104 and the first member 214a may be coupled to the chamber housing 104, all while the chamber 100 remains in the closed configuration. With the first member 214a coupled to the chamber housing 104, the maximum volume of the interior space 102 is greater than it was within the second member 214b coupled to the chamber housing 104. Although described as including third members, the plurality of members 214 may include more than three members.

Referring again to FIGS. 5 and 6, the volume adjustment assembly 210 may be adjustable such that the volume of the interior space 102 is selectable between a range of volumes. As shown in the illustrated embodiment, the volume adjustment assembly 210 may include an adjustment mechanism 216 such as an internally threaded nut. The adjustment mechanism 216 may include a first position in which the volume adjustment assembly 210 defines a minimum volume of the interior space 102. For example, the adjustment mechanism 216 may be tightened all the way down, for example the threaded nut may be bottomed out, such that the member 214 deforms the flexible wall 212 towards the interior space 102 a maximum distance.

The adjustment mechanism 216 may include a second position in which the volume adjustment assembly 210 defines a maximum volume of the interior space 102. For example, the adjustment mechanism 216 may be loosened all the way, for example the threaded nut, the member 212 or both may be removed from the chamber housing 104, such that the member 214 deforms the flexible wall 212 towards the interior space 102 a minimum distance, for example not at all. The inclusion of a volume adjustment assembly 210 allows adjustment of the volume of the interior space 102 while the chamber 100 is in the closed configuration. A chamber 100 capable of varying the volume of the interior space 102 while the chamber 100 is in the closed configuration enables the chamber 100 to adapt to the fetus 2 as the fetus 2 develops, for example the volume of the interior space 102 can be increased to accommodate the increasing size of the fetus 2 as the fetus matures, without the need to remove the fetus 2 from the interior space 102.

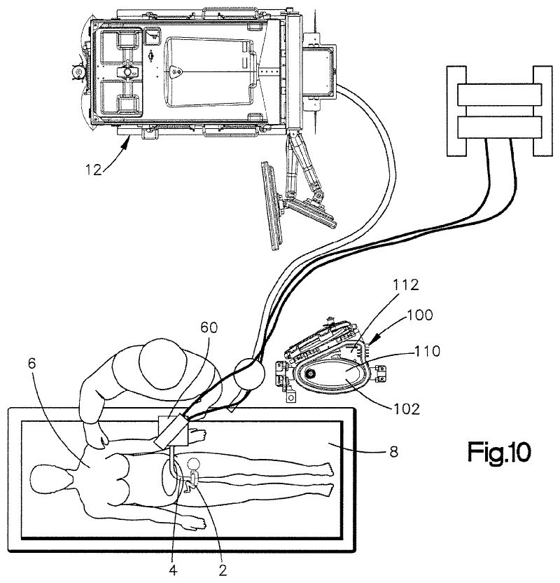

Referring to FIG. 10, a method of moving a premature fetus 2 from the uterus of a patient 6 to an ex-utero environment is provided. The method includes the step of accessing the umbilical cord 4 of the fetus 2. According to one aspect of the disclosure, the accessing step includes making an opening in the uterus of the patient 6 while maintaining uteroplacental perfusion and flow through the umbilical cord. Once the uterus is open and umbilical cord exposed, the method includes the steps of cannulating the umbilical cord vessels (2 arteries and one vein), connecting the cannulas to an oxygenator 60, and then clamping the umbilical cord and severing the umbilical cord 4. The severing step may include the step of separating the fetus from the placenta by clamping and dividing the umbilical cord on the placental side of the cord relative to the cannulas. The connecting step includes the step of attaching the fetus 2 to the oxygenator 60 such that deoxygenated blood is delivered from the fetus 2 to the oxygenator 60, and oxygenated blood is delivered from the oxygenator 60 to the fetus 2. According to one aspect of the disclosure, the method may include, before the attaching step, the step of priming the oxygenator 60, for example with blood.

The step of cannulating the fetus 2 may include the steps of: attaching a first cannula to a vein of the umbilical cord 4, attaching a second cannula to a first artery of the umbilical cord 4, attaching a third cannula to a second artery of the umbilical cord 4, or any combination thereof. The method may further include the step of connecting one or more of the first, second and third cannulae to an oxygenation circuit, which includes the oxygenator 60.

A cannula suitable for use is disclosed in U.S. patent application Publication Number 2021/0338270 titled “Cannula Insertion System And Methods Of Using The Same,” which is incorporated herein by reference in its entirety.

The method further includes the steps of removing the fetus 2 from the uterus of the patient 6 and positioning the fetus 2 within the chamber 100. The method may include the steps of removing the chamber 100 from the cart 12 and positioning the chamber 100 in close proximity to the patient 6, for example on an operating room table 8 upon which the patient 6 is positioned. The system being configured such that the chamber 100 is removable from the cart 12, for example to a location closer to the patient 6 than the cart 12 would be able to go, may reduce the amount of time the fetus 2 is exposed to an ex utero environment during the method, and reduce the potential for contamination, thereby reducing the risk to the fetus 2.

The method further includes, after the cannulating step, the step of positioning the fetus 2 within the chamber interior space 102, and after the positioning step, the step of transitioning the chamber 100 from the open configuration to the closed configuration. The method may further include the step of attaching the chamber 100, with the fetus 2 positioned in the chamber interior space 102, to the cart 12. The method may further include, after the transitioning step, the step of pumping a fluid, for example a sterile fluid, into the chamber interior space 102. The method may include, prior to the pumping step, the step of heating the fluid to a desired temperature, for example a temperature above the ambient room temperature. According to one embodiment, the desired temperature may be in the range of about twenty-eight degrees Celsius to about thirty-eight degrees Celsius, more specifically the desired temperature may be in the range of about thirty degrees Celsius to about thirty-four degrees Celsius. As shown in the illustrated embodiment, the first portion 110 may be configured to receive the fetus 2 and the second portion 112 may be configured to receive at least a portion of the umbilical cord 4 of the fetus 2.

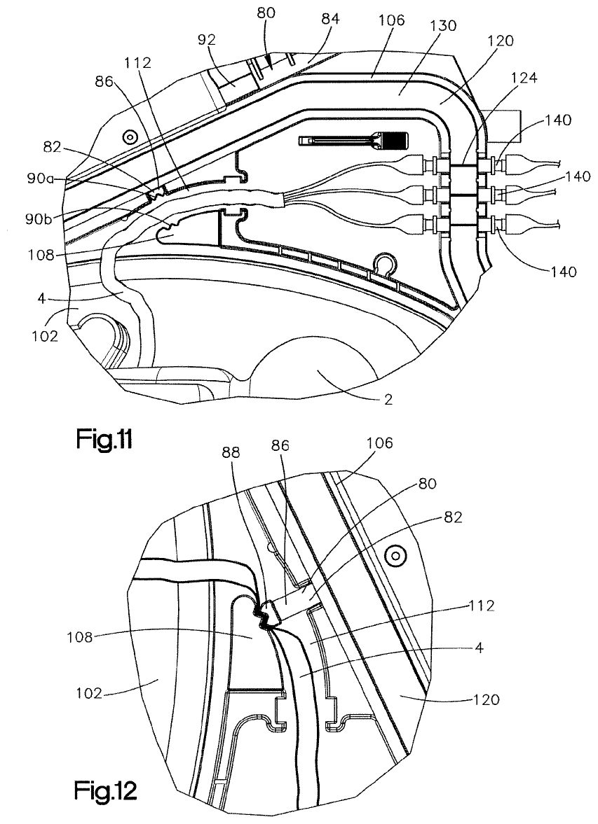

Referring to FIGS. 11 and 12, the system 10 may include a stop assembly 80 configured to clamp the umbilical cord 4 of the fetus 2. According to one aspect of the disclosure, the chamber interior space 102, for example the second portion 112, may be configured to receive a portion of the umbilical cord 4. As shown in the illustrated embodiment the chamber 100 can be configured to receive the umbilical cord 4 between a portion of the outer chamber wall 106 and a portion of the inner chamber wall 108.

As shown in FIGS. 11 and 12, the stop assembly 80 may include a clamp 82 and an actuator 84, the actuator 84 operatively coupled to the clamp 82 such that input, for example by a person operating the system 10, to the actuator 84 transitions the clamp 82 from a first position, illustrated in FIG. 11, also referred to herein as an open position, to a second position, illustrated in FIG. 12, also referred to herein as a closed position. As shown in FIG. 11, in the open configuration the second portion 112 is unobstructed by the clamp 82 such that when the umbilical cord 4 is positioned within the second portion 112 the umbilical cord 4 is unaltered by the clamp 82. As shown in FIG. 12, in the closed configuration the second portion 112 is at least partially obstructed, for example fully obstructed, by the clamp 82 such that when the umbilical cord 4 is positioned within the second portion 112 the umbilical cord 4 is altered, for example clamped such that blood flow through the umbilical cord 4, is prevented.

Prior to placement of the fetus 2 within the chamber interior space 102, at least one cannula 140 may be connected to the fetus 2. As shown a plurality of cannulae 140, for example three cannulae 140, may be connected to the umbilical cord 4 of the fetus 2 such that each of the cannulae 140 is in fluid connection with the circulatory system of the fetus 2. After the umbilical cord 4 of the fetus 2 is cannulated, and the fetus is placed into the chamber interior space 102, one or more of the cannulae 140 may become detached at any time during treatment of the infant 2 such that the one or more of the cannulae 140 are no longer in fluid connection with the circulatory system of the fetus 2, referred to herein as a decannulation event. A decannulation event may pose a risk of serious blood loss for the fetus 2 and thereby a risk to the viability of the fetus 2.

The system 10 may include a decannulation detection assembly configured to detect a decannulation event. According to one embodiment, the system may include a camera configured to detect blood within the interior space 102, which may be an indication of a decannulation event. The camera may be configured to operate and detect blood in the interior space 102 in low light conditions.

The stop assembly 80 is configured to clamp the umbilical cord 4 of the fetus 2 thereby preventing further blood loss after a decannulation event. As shown in the illustrated embodiment, the clamp 82 may include a piston 86 positioned in proximity to, for example within, one of the outer chamber wall 106 and the inner chamber wall 108 when the clamp 82 is in the first position. In the second position the piston 86 extends out from the one of the outer chamber wall 106 and the inner chamber wall 108 toward, for example to, the other of the outer chamber wall 106 and the inner chamber wall 108. According to one aspect of the disclosure, the clamp 82, for example a tip 88 of the piston 86, a portion of the chamber housing 104 that is opposite the clamp 82, or both include an uneven surface 90a and 90b that faces toward the other of the clamp 82 or the portion of the chamber housing 104. The uneven surface 90a and 90b may be configured to provide better clamping of the umbilical cord 4 than an even surface would provide.

The actuator 84 may include a handle 92 operably coupled to the clamp 82 such that actuation, for example rotation, of the handle 92 transitions the clamp 82, for example the piston 86 from one of the first position and the second position to the other of the first position and the second position. The actuator 84 is positioned such that the actuator 84 can be operated to transition the clamp 82 from the first position to the second position while the chamber 100 is in the closed configuration. Thus a method of providing care for the fetus 2, for example the method of moving the fetus 2 from the uterus of a patient 6 to an ex utero environment, may include the step of providing an input to a stop assembly, thereby clamping the umbilical cord 4 of the fetus 2.

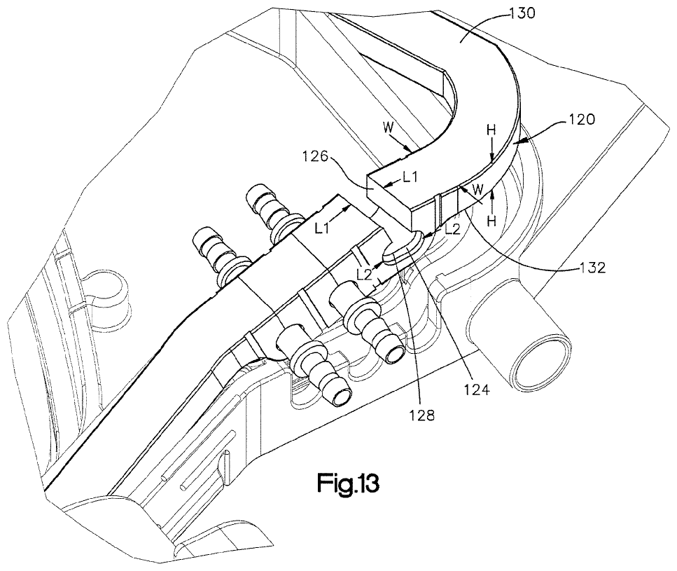

Referring to FIGS. 6, 11, and 13, the chamber housing 104, for example the outer chamber wall 106, includes a first shell 114 and a second shell 116 that are movable relative to one another thereby allowing transition of the chamber 100 from the open configuration to the closed configuration. As shown in the illustrated embodiment, at least one of the first shell 114 and the second shell 116 is rotatable with respect to the other of the first shell 114 and the second shell 116, about at least one hinge 118.

According to one aspect of the disclosure, the chamber includes a seal 120 that includes a resilient material, such that the seal 120 is configured to be compressed between the first shell 114 and the second shell 116 when the chamber 100 is in the closed configuration, and the seal 120 is further configured to expand from the compressed state to an uncompressed state when the chamber 100 is in the open configuration. As shown in the illustrated embodiment, the chamber housing 104, for example the first shell 114, the second shell 116, or both, defines a recess 122 configured to at least partially receive the seal 120. In the closed configuration the seal 120 provides a liquid tight barrier between the chamber interior space 102 and the environment surrounding the chamber 100.

According to one aspect of the disclosure, the seal 120 defines a height H that is measured between the first shell 114 and the second shell 116 when the chamber 100 is in the closed configuration, and the seal further defines a width W that is perpendicular to the height H. As shown in the illustrated embodiment, the width W may be measured between the chamber interior space 102 and the environment surrounding the chamber 100.

The seal 120 defines at least one slot 124 configured to receive at least one of the cannulae 140 that are connected to the fetus 2. The slot 124 may include a first portion 126 that defines a first length L1 that is perpendicular to both the height H and the width W. The slot may include a second portion 128 that defines a second length L2 that is perpendicular to both the height H and the width W. Both the first length L1 and the second length L2 may be measured from one side wall of the seal 120 to an opposing side wall of the seal 120 that faces the one side wall. According to one aspect of the disclosure the first length L1 is less than the second length L2.

As shown in the illustrated embodiment, the first portion 126 of the slot 124 may extend from a first outer surface 130 of the seal 120 toward a second outer surface 132 of the seal 120 that is opposite the first outer surface 130 of the seal 120. The first outer surface 130 and the second outer surface 132 may each be surfaces that the height His normal to. The seal 120 may terminate prior to reaching the second outer surface 132 of the seal 120. As shown the first portion 126 is positioned between the first outer surface 130 and the second portion 128 with respect to the direction the height H is measured along.

The slot 124 may be configured to receive the cannula 140 when the chamber 100 is in the open configuration. The cannula 140 may be moved through the first portion 126 of the slot 124 and then positioned within the second portion 128 of the slot 124. When the cannula 140 is positioned within the second portion 128, transitioning the chamber 100 into the closed configuration causes both the first portion 126 to form a liquid tight barrier and the second portion 128 to form a liquid tight barrier around the cannula 140. As shown in the illustrated embodiment, the seal 120 may define three of the slots 124 arranged such that the slots 124 extend through the seal 120 substantially perpendicular to one another. The seal 120 may include other numbers of the slots 124, for example less than three or more than three, and other arrangements of the slots 124, for example non-parallel to one another. As shown in the illustrated embodiment, the slot 124 may face the second portion 112 of the chamber interior space 102.

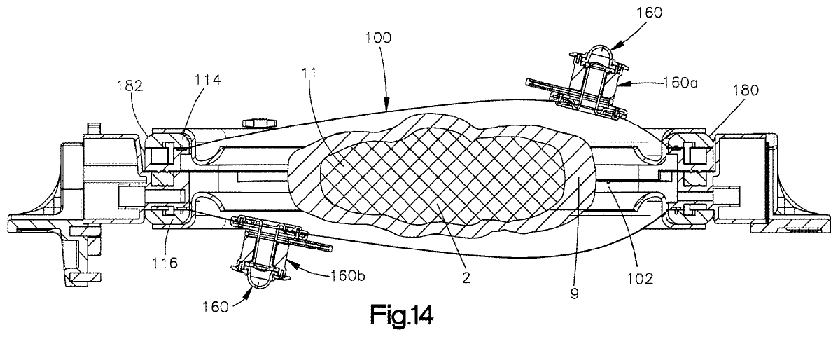

Referring to FIG. 14, the chamber 100 may include a port 160 configured to provide a passageway from the environment surrounding the chamber 100 to the chamber interior space 102 when the chamber 100 is in the closed configuration. As shown in the illustrated embodiment the chamber 100 may include a plurality of ports 160 including a first port 160a and a second port 160b. The first port 160a and the second port 160b may be positioned opposite one another. For example, the first port 160a may be supported by the first shell 114 and the second port 160b may be supported by the second shell 116. The first port 160a may be positioned closer to the first end 180 of the chamber 100 than the second port 160b is to the first end 180, and the second port 160b may be positioned closer to the second end 182 of the chamber 100 than the first port 160a is to the second end 182. As shown in the illustrated embodiment, the first port 160a and the second port 160b may be positioned such that when the fetus 2 is in the chamber 100 and the chamber 100 is in the closed configuration, the first port 160a is positioned proximate the head 7 of the fetus 2 and the second port 160b is positioned proximate the feet 9 of the fetus 2. The chamber 100 including the first port 160a proximate the fetus' head and the second port 160b proximate the fetus' feet provides selectable access to the chamber interior space 102 to remove debris from the chamber interior space 102 that is positioned either by the head 7 or feet 9 of the fetus 2.

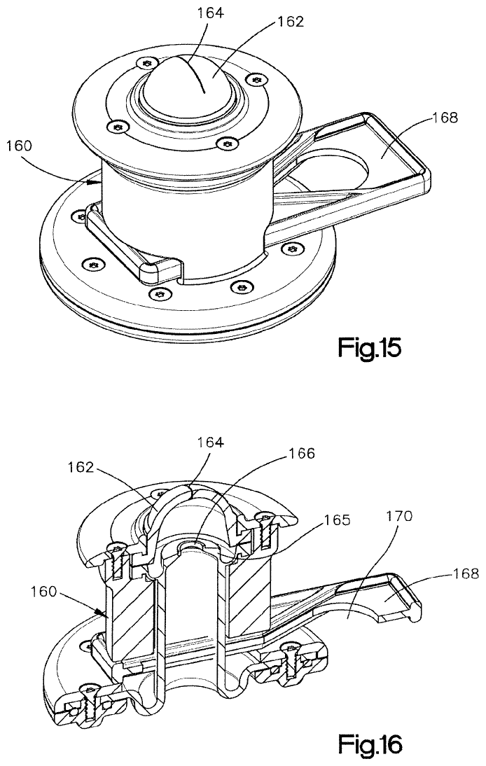

Referring to FIGS. 15 to 18, the port 160 is configured to provide access for an instrument 200, for example a suction wand 202, into the chamber interior space 102 while maintaining sterility of the chamber interior space 102. The port 160 may include a first seal 162 that is biased closed. As shown in FIGS. 15 to 17, when the instrument 200 is removed from the port 160, a slit 164 of the first seal 162 is biased closed. As shown in FIG. 18, the port 160 may include a second seal 165 that is configured to form a seal around the instrument 200 when the instrument is inserted into the port 160. According to one embodiment, the second seal 165 is spaced from the first seal 162, and the second seal 165 defines an opening 166. The opening 166 may correspond to a shape, for example match a shape, of an exterior surface of the instrument 200, such that when the instrument 200 is inserted into the port 160, the opening 166 provides a passageway for the instrument 200 and forms a seal with the instrument 200.

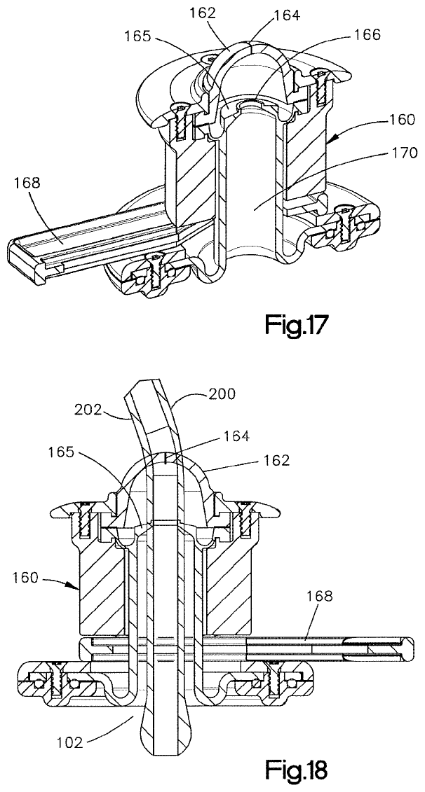

According to one aspect of the disclosure, the port 160 may include a third seal 168 that is moveable from a first position to a second position. As shown in FIGS. 15 and 16, in the first position, also referred to herein as a closed position, the third seal 168 blocks the passageway through the port 160, such that the instrument 200 cannot pass through the port 160 into the chamber interior space 102. As shown in FIGS. 17 and 18, the third seal 168 may be moved, for example translated, such that an opening 170 of the third seal 168 is aligned with the passageway of the port 160, and the instrument 200 can pass through the port 160 into the chamber interior space 102.

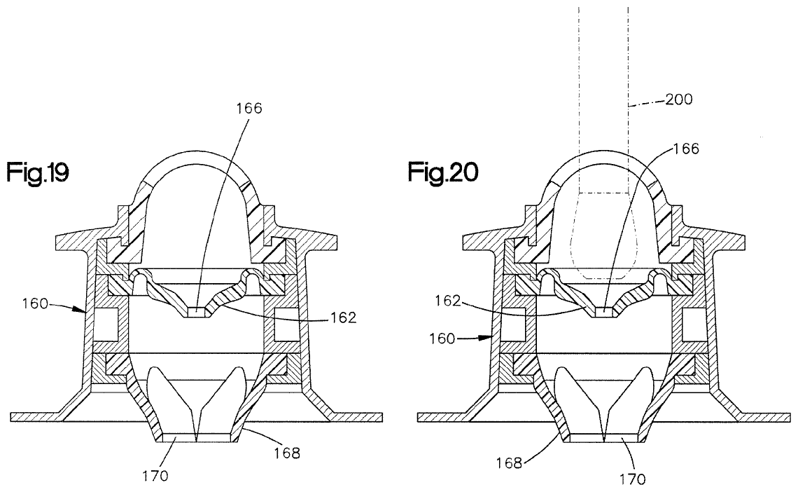

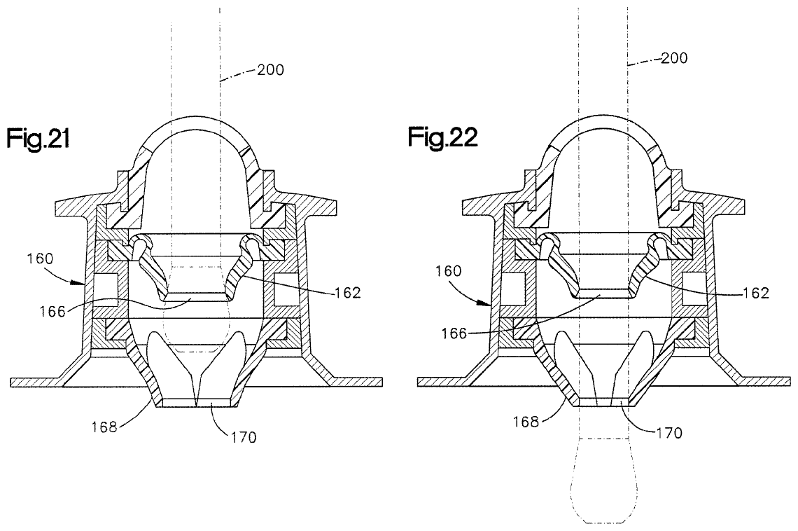

Referring to FIGS. 19 to 22, according to another embodiment, the third seal 168 of the port 160 may be similar to the second seal 164 as described above in reference to FIGS. 15 to 18, such that the third seal 168 is not movable from a first position to a second position, but rather the opening 170 is fixed in position to the passageway of the port 160 and corresponds to a shape of the instrument 200, such that when the instrument 200 is inserted into the port 160, the opening 166 provides a passageway for the instrument 200 and forms a seal with the instrument 200. As shown in the illustrated embodiment, the opening 170 may be larger than the opening 166 when instrument 200 is removed from the port 160.

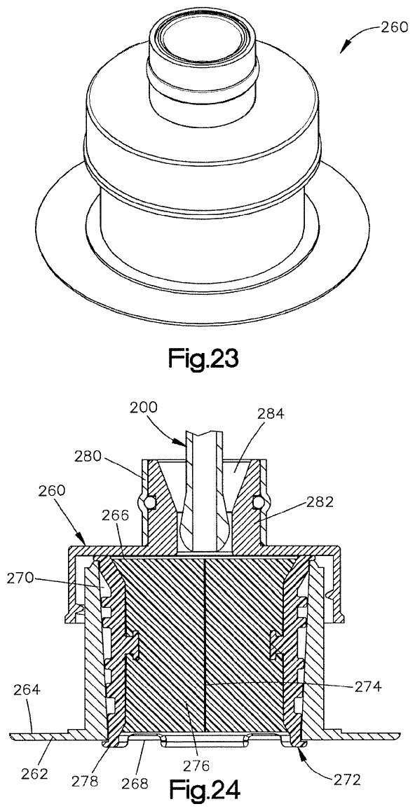

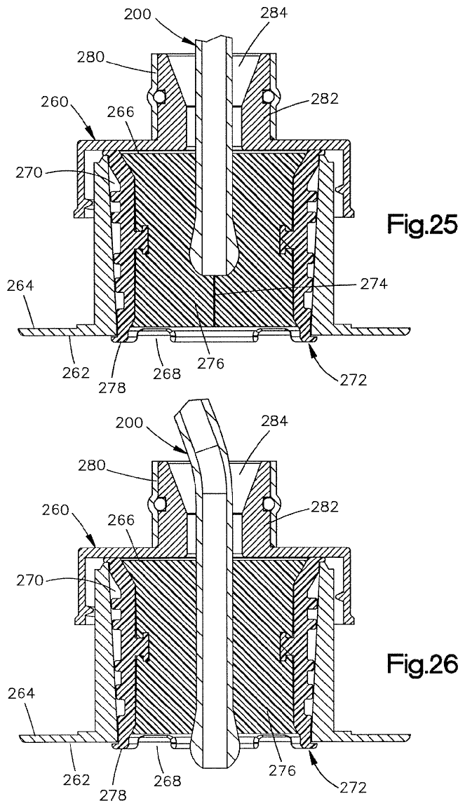

Referring to FIGS. 23 to 26, the system 10 may include a port 260 instead of or in addition to the port 160. The port 260 may include a housing 262 configured to be attached to one of the first shell 114 and the second shell 116. For example, an upper surface 264 of the housing 262 may be welded to one of the flexible wall 212a and 212b, for example the side of the flexible wall 212a and 212b that faces the interior space 102. Alternatively, the housing 262 may be configured to be welded to the side of the flexible wall 212a and 212b that faces away from the interior space 102. The housing 260 defines a first opening 266, a second opening 268 and a recess 270 that extends from the first opening 266 to the second opening 268.

The port 260 further includes an insert 272 positioned within the recess 270. The insert 272 is configured to create a seal, for example a liquid-tight seal, an air-tight seal, or both, between the first opening 266 and the second opening 268. The insert 272 includes an elastically deformable material with a slit 274. The insert 272 is configured to allow the instrument 200 to be inserted into the slit 274 and form a seal around the instrument 200 as the instrument is inserted through the slit 274. The insert 272 may include a first material 276, for example silicone, configured to compress as the instrument 200 is inserted into the slit 274 and apply a biasing force against the instrument 200 thereby maintaining a seal between the first opening 266 and the second opening 268.

The insert 272 may further include a second material 278, for example a polycarbonate, that is stiffer than the first material 276. The second material 278 may be positioned around the first material 276 and may include an attachment mechanism to secure the insert 272 within the recess 270.

The port 260 may also include a cap assembly 280. The cap assembly 280 includes a body 282 that defines an opening 284 configured to guide the instrument to the slit 274. The cap assembly 280 may be attached to the housing 260, for example threadedly attached with corresponding threads.

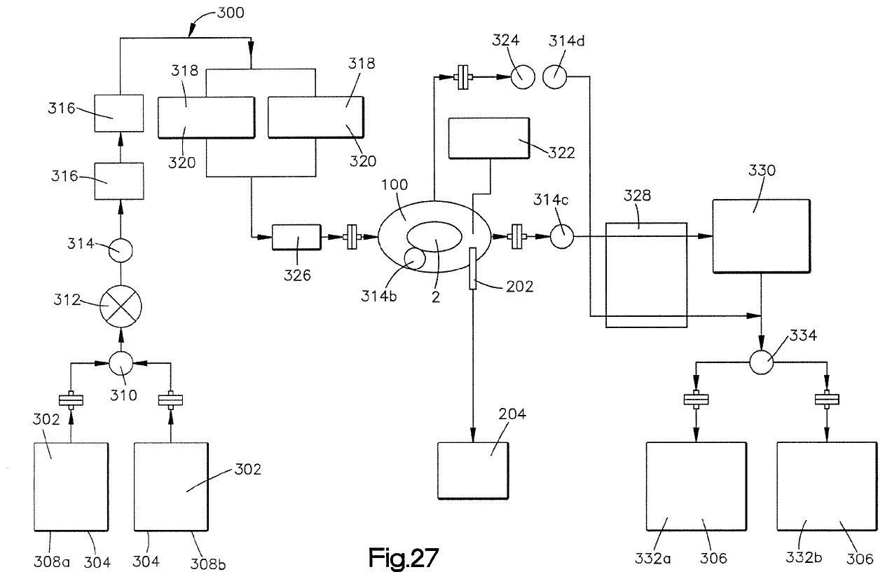

Referring to FIG. 27, the system 10 includes a first fluid circuit 300, which is configured to deliver a fluid 302 from a source 304 to the chamber 100, and then deliver the fluid 302 from the chamber 100 to a reservoir 306. The fluid 302 may be a sterile solution, for example an electrolyte solution. In one embodiment, fluid 302 comprises a physiological saline solution such as solutions described in pending application U.S. Ser. No. 17/970,503, which is incorporated herein by reference in its entirety. The source 304 may include multiple source containers, for example a first source container 308a and a second source container 308b. The multiple source containers may be arranged in parallel such that the fluid 302 can be delivered from one or another of the multiple source containers. According to one aspect of the disclosure, the first fluid circuit 300 includes a valve 310 that is configured to provide passage of the fluid 302 from the source 304 to the chamber 100 when the valve 310 is in an open configuration, and the valve 310 is further configured to block passage of the fluid 302 from the source 304 to the chamber 100 when the valve 310 is in a closed configuration.

As shown in the illustrated embodiment, the valve 310 may be a three-way valve that includes a first open configuration in which the valve 310 provides passage of the fluid 302 from the first source container 308a to the chamber 100 while blocking passage of the fluid 302 from the second source container 308b to the chamber 100. The three-way valve may further include a second open configuration in which the valve 310 provides passage of the fluid 302 from the second source container 308b to the chamber 100 while blocking passage of the fluid 302 from the first source container 308a to the chamber 100. The valve 310 being a three-way valve as described above would allow for the fluid 302 from the first source container 308a to be delivered to the chamber 100 until the first source container 308a is empty, then the valve 310 could be transitioned from the first open configuration to the second open configuration allowing the fluid 302 from the second source container 308b to be delivered to the chamber 100, while allowing the, now empty, first source container 308a to be replaced with a new container.

The first fluid circuit 300 includes a pump 312, for example a peristaltic pump, configured to move the fluid 302 from the source 304 to the chamber 100. The first fluid circuit 300 may include a first pressure sensor 314a positioned between the pump 312 and the chamber 100, a second pressure sensor 314b positioned within the chamber 100, a third pressure sensor 314c positioned between the chamber 100 and the reservoir 306, a fourth pressure sensor 314d, or any combination thereof. Each of the pressure sensors 314a, 314b, 314c, and 314d may be configured to output a numerical value representing the current pressure within the first fluid circuit 300 between the pump 312 and the chamber 100 to a display viewable by a user of the system 10.

The first fluid circuit 300 may include a filters 316 configured to block particulates in the fluid 302 from reaching the chamber 100. The filter 316 may be configured to block particulates of a selected size, for example particles greater than about 0.22 micrometers. The filter 316 may be one of a plurality of filters 316 that can be arranged in parallel or in series. The plurality of filters 316 may be configured to block particulates of the same size, or of different sizes. For example a first of the plurality of filters 316 may be configured to block particulates of a first size, and a second of the plurality of filters 316 may be configured to block particulates of a second size. The first size may be larger than the second size, and the second of the plurality of filters 316 may be positioned between the first of the plurality of filters 316 and the chamber 100.

The first fluid circuit 300 may include a heat source 318 configured to change a temperature of the fluid 302 prior to reaching the chamber 100. The heat source 318 may include one or more heaters 320 configured to increase the temperature of the fluid 302. The first fluid circuit 300 may include a turbidity meter 322, configured to measure the clarity of the fluid 302. The turbidity meter 322 may be positioned within the chamber 100 and configured to send a signal, for example activate an alarm of the system 10, when a level of cloudiness is present within the fluid 302. Cloudiness in the fluid 302 may be caused by a contaminate in the fluid 302 in the chamber 100, for example meconium. The turbidity meter 322 may be configured to detect blood in the chamber 100. The presence of blood in the chamber 100 may signal a decannulation event, which may require rapid detection and notification to minimize potential harm to the infant 2.

The system 10 is configured to facilitate removal of the contaminate in the fluid 302 in the chamber 100 while maintaining the chamber 100 in the closed configuration. For example the suction wand 202 may be inserted into the chamber 100, for example through one of the ports as described above, and used to remove the contaminate. The suction wand 202 may be connected to a vacuum source 204, for example a mobile vacuum source or a fixed vacuum source.

The first fluid circuit 300 may include a release valve 324 configured to provide release the fluid 302 within the chamber 100 more quickly than the fluid 302 would normally exit the chamber 100 toward the reservoir 306. If the pressure of the fluid 302 within the chamber 100 reaches a value above a desired level, or if quick access to the fetus 2 is desired, actuation of the release valve 324 will empty the chamber 100 of the fluid 302 currently within the chamber 100.

The first fluid circuit 300 may include a flow meter 326 configured to measure a rate at which the fluid 302 is moving through the first fluid circuit 300. The first fluid circuit 300 may include a first flow meter 326 positioned between the pump 312 and the chamber 100 such that the first flow meter 326 is configured to measure the flow rate of the fluid 302 into the chamber 100, a second flow meter 326 positioned between the chamber 100 and the reservoir 306 such that the second flow meter 326 is configured to measure the flow rate of the fluid 302 out of the chamber 100, or both.

The first fluid circuit 300 may include a filtration system 328 positioned between the chamber 100 and the reservoir 306. The filtration system 328 is configured to prevent contaminates, such as bacteria, from migrating toward the chamber 100 along a direction that is opposite the direction of flow of the fluid 302. According to one aspect of the disclosure, the filtration system 328 is configured to kill bacterial growth that migrates from the reservoir 306 toward the chamber 100.

The first fluid circuit 300 may include a pressure regulator 330 configured to adjust the pressure of the fluid 302 within the chamber 100. The pressure regulator 330 may include an actuator that is configured to receive an input, for example a manual input that includes raising or lowering the actuator with respect to the surface the system 10 is positioned upon, to raise or lower, respectively, the pressure of the fluid 302 within the chamber 100.

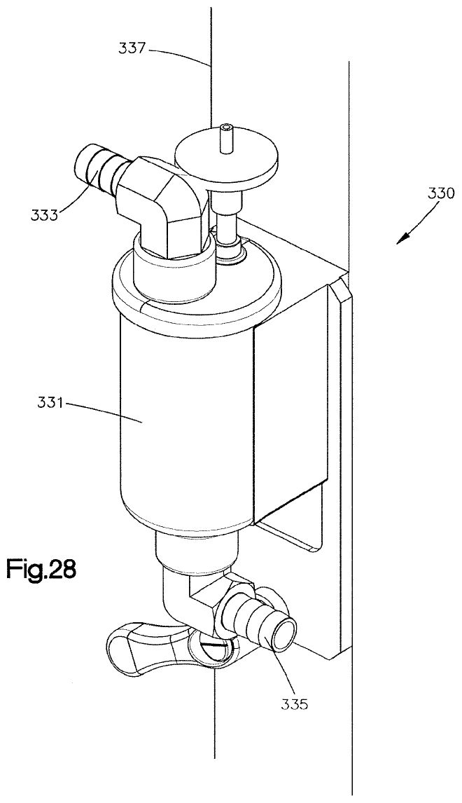

Referring to FIGS. 27 and 28, the pressure regulator 330 may include a pressure chamber 331, a first port 333 coupled to the pressure chamber 331 and a second port 335 coupled to the pressure chamber 331. The pressure regulator 330 may be configured such that the fluid 302 discharged from the chamber 100 enters the pressure chamber 331 through the first port 333 and exits through the second port 335 on the way towards the reservoir 306. As shown in the illustrated embodiment, the pressure chamber 331 is slidably mounted to the cart 12, for example on a pair of rails 337. According to one aspect of the disclosure, the pressure inside the interior space 102 may be adjusted by adjusting the height of the pressure chamber 331 relative to the interior space 102. For example, the system 10 may be configured such that by sliding the pressure chamber 331 “up” along the rails 337 thereby increasing the height of the pressure chamber 331 relative to the interior space 102, the fluid 302 exiting the interior space 102 must travel “up” against gravity, thereby increasing the pressure within the interior space 102. The system 10 may further be configured such that by sliding the pressure chamber 331 “down” along the rails 337 thereby decreasing the height of the pressure chamber 331 relative to the interior space 102, the fluid 302 exiting the interior space 102 has less of a vertical distance to travel “up” against gravity, thereby decreasing the pressure within the interior space 102.

The reservoir 306 may include multiple reservoir containers, for example a first reservoir container 332a and a second reservoir container 332b. The multiple reservoir containers may be arranged in parallel such that the fluid 302 can be delivered from one or another of the multiple reservoir containers 332a and 332b. According to one aspect of the disclosure, the first fluid circuit 300 includes a valve 334 that is configured to provide passage of the fluid 302 from the chamber 100 to the reservoir 306 when the valve 334 is in an open configuration, and the valve 334 is further configured to block passage of the fluid 302 from the chamber 100 to the reservoir 306 when the valve 334 is in a closed configuration.

As shown in the illustrated embodiment, the valve 334 may be a three-way valve that includes a first open configuration in which the valve 334 provides passage of the fluid 302 from chamber 100 to the first reservoir container 332a while blocking passage of the fluid 302 from the chamber 100 to the second reservoir container 332b. The three-way valve may further include a second open configuration in which the valve 334 provides passage of the fluid 302 from the chamber 100 to the second reservoir container 332b while blocking passage of the fluid 302 from the chamber 100 to the first reservoir container 332a. The valve 334 being a three-way valve as described above would allow for the fluid 302 from the chamber 100 to be delivered to the first reservoir container 332a until the first reservoir container 332a is full, then the valve 334 could be transitioned from the first open configuration to the second open configuration allowing the fluid 302 from the chamber 100 to be delivered to the second reservoir container 332b, while allowing the, now full, first reservoir container 332a to be removed and replaced with a new, empty container.

Referring to FIGS. 27 and 29 to 30, the filtration system 328 may include an ultraviolet light source 360 configured to deliver an amount of ultraviolet light to the fluid 302 between the chamber 100 and the reservoir 306. Bacteria or other contaminants may grow within the reservoir 306 and grow or migrate toward the chamber 100 retrograde, or opposite the flow of the fluid 302. The filtration system 328 is configured to eradicate the contaminate before the contaminate reaches the chamber 100.

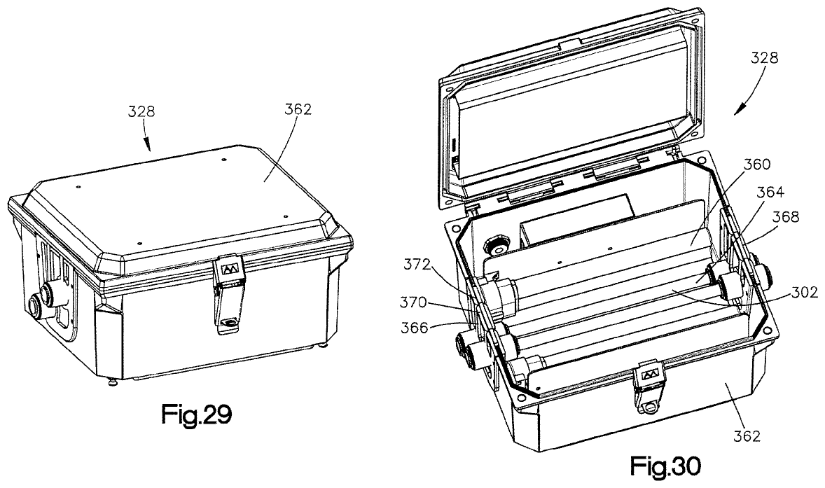

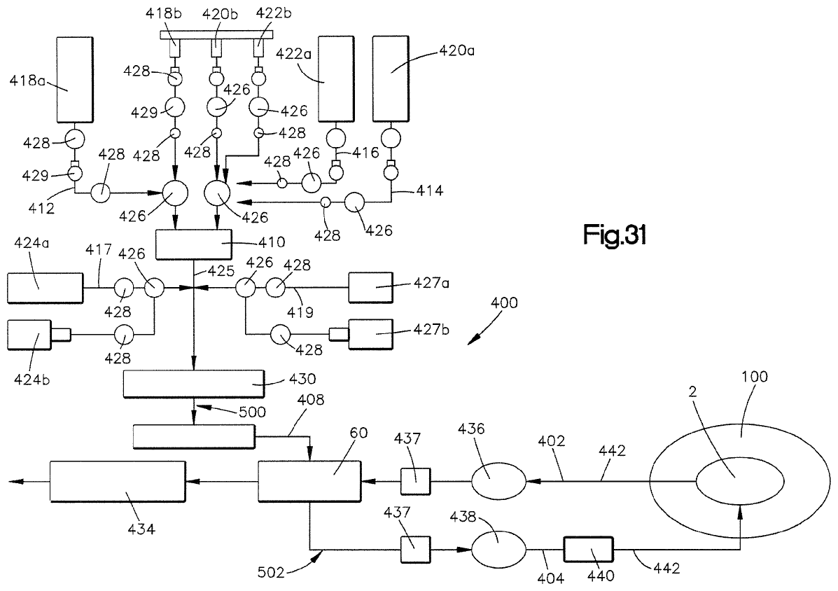

The filtration system 328 may include a housing 362 configured to limit an amount of ultraviolet light that exits the filtration system 328. According to one embodiment, the housing 362 defines an open configuration (as shown in FIG. 30) and a closed configuration (as shown in FIG. 29). In the closed configuration the housing 362 is configured to block a portion, for example all, of the ultraviolet light from reaching the chamber 100. The filtration system 328 may include a length of tubing 364 that is exposed to the ultraviolet light source 360 and that carries the fluid 302 between the chamber 100 and the reservoir 306.

The housing 362 may include a first seal 366 and a second seal 368 that are each configured to receive the tubing 364 such that when the tubing 364 is positioned within the first seal 366 and the second seal 368, the first seal 366 and the second seal 368 form a light barrier around the tubing 364 preventing the ultraviolet light from exiting the housing 362. The first seal 366, the second seal 368, or both may include a slot 370 extending from an outer surface 372 in a direction substantially perpendicular to the direction of flow of the fluid 302 within the tubing 364. The slot 370 may be configured to facilitate sliding engagement of the tubing 364 with the respective seal. The housing 362 may include a reflective surface 374 positioned within the housing 362 such that the reflective surface 374 is configured to reflect the ultraviolet light to additional areas of the tubing 364 that are not directly exposed to the ultraviolet light source 360.

The filtration system 328 defines a length L3 measured along the section of tubing that is exposed to the ultraviolet light and measured in the direction of the flow of the fluid 302. According to one aspect of the disclosure, the length L3 is greater than about 0.8 inches, the irradiance provided by the ultraviolet light source 360 is about 100 microwatts per centimeter squared, the cross-sectional area of the tubing 364 is about 0.22 centimeters squared, and the flowrate of the fluid 302 through the tubing 364 may be up to about 32 milliliters per minute.

Thus a method of providing care for the fetus 2, for example the method of moving the fetus 2 from the uterus of a patient 6 to an ex utero environment, may include the step of exposing a portion of a first fluid circuit 300 that flows through the chamber 100 to ultraviolet light.

Referring to FIG. 31, the system 10 includes a second fluid circuit 400 configured to provide gas transfer between the fetus 2 and the oxygenator 60. Specifically, the second fluid circuit 400 is configured to provide oxygen to and remove carbon dioxide from the blood of the fetus 2. The second fluid circuit 400 may include a first portion 500 configured to deliver a sweep gas 408 to the oxygenator 60, and a second portion 502 configured to accept the sweep gas 408 and perform gas exchange with the blood supply of the fetus 2. The second portion 502 may include the oxygenator 60 connected with the fetus 2 by two fluid lines of the second fluid circuit 400. The two fluid lines include an outflow line 402 and an inflow line 404. The blood of the fetus 2 flows from the fetus 2 though the outflow line 402 to the oxygenator 60, the blood then flows through the oxygenator 60 and returns to the fetus 2 through the inflow line 404. The outflow line 402 and the inflow line 404 pass through the seal 120, for example by way of the cannulae 140.

The system 10 may be configured such that the oxygenator 60 is positioned close to the chamber 100 such that the lengths of the outflow line 402 and the inflow line 404, to and from the oxygenator 60 respectively, are minimized. For instance, in accordance with one aspect of the disclosure, the outflow line 402 and the inflow line 404 are less than about 36 inches long combined. By minimizing the lengths of the outflow line 402 and the inflow line 404, the volume of blood required to prime the second fluid circuit 400 is minimized. It may be desirable to line the outflow line 402 the inflow line 404, or both with anti-clotting measures/compounds (for example, but not limited to, immobilized polypeptide, heparin, or both).

The oxygenator 60 may be primed prior to connection with the fetus 2. According to one embodiment, the oxygenator 60 may be primed with a crystalloid solution containing human albumin. The second fluid circuit 400 may then be further primed with, for example, maternal blood, blood of the fetus 2, or both. Priming of the second fluid circuit 400 with hemoglobin from the fetus 2 may result in optimal oxygen exchange in the second fluid circuit 400. Because the fetal oxygen dissociation curve is shifted to the left compared to the adult oxygen dissociation curve, fetal arterial oxygen pressures are lower than adult arterial oxygen pressures. In a one embodiment, the blood in the second fluid circuit 400 includes heparin. According to another embodiment, the blood in the second fluid circuit 400 is devoid of heparin.

According to one aspect of the disclosure, the first portion 500 of the second fluid circuit 400 produces a sweep gas 408 and delivers the sweep gas 408 to the oxygenator 60, the sweep gas 408 is configured to facilitate gas transfer between the oxygenator 60 and the blood of the fetus 2. The gas transfer is affected by the composition of the sweep gas 408 and the flow rate of the sweep gas 408 through the oxygenator 60. A plurality of gases may be blended together in a gas blender 410 that blends the plurality of gases to form the sweep gas. According to one aspect of the disclosure, the plurality of gases may include, but is not limited to, oxygen, nitrogen, carbon dioxide, nitric oxide, air, or any combination thereof. As shown in the illustrated embodiment, the plurality of gases may include at least a first gas 412 and a second gas 414. The plurality of gases may further include a third gas 416, a fourth gas 417, a fifth gas 419, or any combination thereof. According to one aspect of the disclosure, any or all of the plurality of gases may be supplied by multiple sources such as a mobile, smaller source, and a larger, fixed source.

As an example, the second fluid circuit 400 may include a mobile first gas source 418a, for example that is attached to and carried by the cart 12, and a fixed first gas source 418b that is fixed in place, for example as part of the infrastructure of a hospital room. The second fluid circuit 400 may further include a mobile second gas source 420a, a fixed second gas source 420b, a mobile third gas source 422a, a fixed third gas source 422b, a mobile fourth gas source 424a, a fixed fourth gas source 424b, a mobile fifth gas source 427a, a fixed fifth gas source 427b, or any combination thereof.

The first portion 500 of the second fluid circuit 400 may include a plurality of valves 426 each configured to control whether the mobile or fixed source of each respective gas source is connected with the second fluid circuit 400. The second fluid circuit 400 may include one or more pressure sensors 428 positioned inline with each of the plurality of gas supplies, the plurality of pressure sensors 428 configured to measure the gas pressure of the plurality of gases being fed to the second fluid circuit 400. The second fluid circuit 400 may further include one or more pressure regulators 429 configured to provide receive a variable pressure from the respective gas source and deliver a steady, constant pressure of the respective gas. As shown in the illustrated embodiment, the pressure regulator 429 may be positioned between two of the pressure sensors 428, which may be configured to measure the pressure going into and coming out of the pressure regulator 429.

The second fluid circuit 400 may be configured such that one or more of the plurality of gases enter the gas blender 410. As shown in the illustrated embodiment, the first gas 412 and one of the second gas 414 and the third gas 416 may enter the gas blender 410. According to one aspect of the disclosure, the first gas 412 is oxygen, the second gas 414 is nitrogen, and the third gas 416 is air. The gas blender 410 outputs a mixed gas 425. The second fluid circuit 400 may be configured such that one or more of the plurality of gases combines with the mixed gas 425 after the mixed gas 425 exits the gas blender 410. According to one embodiment, the plurality of gases includes oxygen, nitrogen, and air coupled to the second fluid circuit 400 such that the oxygen and one of nitrogen and the air enter the gas blender 410 to form the mixed gas 425. The second fluid circuit 400 may include the fourth gas 417, the fifth gas 419, or both connected to the mixed gas 425 to form the sweep gas 408. The fourth gas may include carbon dioxide and the fifth gas may include nitric oxide, according to one aspect of the disclosure that are each configured to be added to the mixed gas 425 after the mixed gas 425 exits the gas blender 410, to form the sweep gas 408.

The second fluid circuit 400 may include a heater, for example the heater 42, or a different heater that is positioned inline between the gas blender 410 and the oxygenator 60, the heater configured to heat the sweep gas 408 so that the temperature of the sweep gas 408 is maintained within a predetermined range. The second fluid circuit 400 may include a fluid flow regulator 430 configured to monitor, adjust, or both the flow rate of the sweep gas 408. The second fluid circuit 400 may further include a sweep gas analyzer 432 configured to analyze one or more characteristics of the sweep gas 408 entering the oxygenator 60.

The second fluid circuit 400 may include an exhaust gas analyzer 434 configured to analyze one or more characteristics of the gas discharged by the oxygenator 60. For instance, the gas analyzers 432 and 434 may be configured to measure the oxygen content of the sweep gas and the exhaust gas, respectively. As shown in the illustrated embodiment, the fluid flow regulator 430 may be positioned between the sweep gas analyzer 432 and the oxygenator 60.

The second fluid circuit 400 further includes a pair of fluid pressure sensors 436 and 438 configured to detect the fluid pressure of the blood entering the oxygenator 60 and the fluid pressure of the blood exiting the oxygenator 60, respectively. Specifically, the first pressure sensor 436 may be positioned in-line with the outflow line 402 and the second pressure sensor 438 may be positioned in-line with the inflow line 404. In this way, the fluid pressure drop over the oxygenator 60 can be continuously monitored. Additionally, a fluid flow meter 440 may be positioned in-line with the inflow line 404 to monitor the flow rate of the blood returning to the fetus 2 from the oxygenator 60.

The second fluid circuit 400 may include one or more ports 442, which may be utilized to withdraw blood samples for analysis or the ports 442 may be used to inject or infuse medicine or nutrition directly into the blood. For instance, one of the ports 442 may be configured to facilitate injection of medication such as antibiotics or sedatives into the blood. Similarly, another of the ports 442 may be configured to facilitate injection of nutrition such as total parental nutrition (TPN) into the blood.

The second portion 502 of the second fluid circuit 400 may include a first sensor 437 and a second sensor 439 positioned such that the oxygenator 60 is between, for example directly between, the first sensor 437 and the second sensor 439. The first sensor 437 and the second sensor 439 may configured to measure one or more variables of the blood of the fetus 2 just before entering the oxygenator 60 and just after exiting the oxygenator 60, respectively, so that the change provided by the oxygenator can be measured and monitored. The first sensor 437 and the second sensor 439 may each be configured to measure blood flow, blood oxygen levels, blood hemoglobin levels, or any combination thereof. According to one embodiment, the first sensor 437 and the second sensor 439 each use absorbance spectroscopy to measure the amount of oxygen bound to hemoglobin, and levels of hemoglobin. Blood flow may be measured by ultrasound.

In accordance with one aspect of the disclosure, the heart of the fetus 2 drives blood flow through the second portion 502 of the second fluid circuit 400, such that the second portion 502 of the second fluid circuit 400 is devoid of a pump. In other words, according to one aspect of the disclosure, the second portion 502 of the second fluid circuit 400 is a pumpless circuit. The use of a pumpless system avoids exposure of the heart of the fetus 2 to excess preload encountered in non-pulsatile pump-assisted circuits. The pumpless system also permits intrinsic fetal circulatory regulation of flow dynamics. The oxygenator 60 preferably has very low resistance, low priming volume, low transmembrane pressure drops, and provides efficient gas exchange. The first portion 500 of the second fluid circuit 400 may be driven by an external pressure source.

In accordance with one embodiment, the oxygenator 60 has a pressure drop of less than about 50 mmHg or about 40 mmHg at 1.5 liters per minute of blood flow. In a particular embodiment, the priming volume of the oxygenator 60 is less than about 100 milliliters and in particular is less than about 85 milliliters. In a particular embodiment, the oxygenator 60 has a blood flow range up to about 2.0 liters per minute, about 2.5 liters per minute, about 2.8 liters per minute, or greater. In a particular embodiment, the oxygenator 60 has a gas transfer rate of about 150 milliliters per minute, about 160 milliliters per minute, about 180 milliliters per minute, or greater for oxygen. In a particular embodiment, the oxygenator 60 is a hollow fiber membrane oxygenator (for example, but not limited to, a polymethyl pentene hollow fiber membrane oxygenator). The oxygenator 60 may be lined with anti-clotting measures/compounds such as immobilized polypeptide and/or heparin). An exemplary embodiment of oxygenator 60 is shown in pending application PCT US 2022/043259, which is incorporated herein by reference in its entirety.

The system 10 may be configured for use with fetuses, including term and preterm fetuses. The preterm fetus may be a premature fetus (for example, less than 37 weeks estimated gestational age, particularly 28 to 32 weeks estimated gestational age), extreme premature fetuses (24 to 28 weeks estimated gestational age), or pre-viable fetuses (20 to 24 weeks estimated gestational age). The gestation periods are provided for humans, though corresponding preterm fetuses of other animals may be used. In a particular embodiment, the preterm fetus has no underlying congenital disease. In a particular embodiment, the term or preterm fetus has limited capacity for pulmonary gas exchange, for example, due to pulmonary hypoplasia or a congenital anomaly affecting lung development, such as congenital diaphragmatic hernia. In a particular embodiment, the subject is a preterm or term neonate awaiting lung transplantation, for example, due to congenital pulmonary disease (e.g., bronchoalveolar dysplasia, surfactant protein B deficiency, and the like). Such transplantation surgeries are currently rarely performed in the United States. However, the number of transplantation surgeries may be increased with the more stable method for pulmonary support provided by the instant invention. The fetus 2 may also be a candidate for ex utero intrapartum treatment (EXIT) delivery, including patients with severe airway lesions and a long expected course before definitive resection. The fetus 2 may also be a fetal surgical or fetoscopic procedure patient, particularly with preterm labor precipitating early delivery. According to one aspect of the disclosure the system 10 is configured such that the fetus 2 may be maintained in the system 10 for as long as needed (for example, for days, weeks or months, until the fetus 2 is capable of life without the system 10).

In another aspect, a method is provided for treating a premature fetus by reducing a level of bilirubin in a blood of a premature fetus in an ex-utero environment, the method comprising the steps of a) connecting a premature fetus to an ex-utero system configured to provide oxygen to the fetus, wherein the connecting step comprises the steps of attaching a first cannula to a vein of the umbilical cord, attaching a second cannula to a first artery of the umbilical cord, attaching a third cannula to a second artery of the umbilical cord, and connecting one or more of the first, second and third cannulae to the oxygenator via an oxygenation circuit, wherein at least one of the cannulae is in fluid connection with the fetus' circulatory system and the umbilical cord is connected to the oxygenator such that deoxygenated blood is delivered from the fetus to the oxygenator, and oxygenated blood is delivered from the oxygenator to the fetus, and wherein the system further comprises a port in fluid connection with the fetus' circulatory system; and b) exposing at least a portion of the oxygenation circuit is exposed to a phototherapy device having a light output ranging from about 430 nanometers (nm) to about 490 nm. In some embodiments, the port is located on the oxygenation circuit downstream from the oxygenator.

In another aspect, a method is provided for extracorporeally treating hyperbilirubinemia in a premature fetus' blood in an ex-utero environment, the method comprising the steps of: (a) connecting a premature fetus to an ex-utero system as disclosed above and configured to provide oxygen to the fetus, wherein the connecting step comprises the steps of attaching a first cannula to a vein of the umbilical cord, attaching a second cannula to a first artery of the umbilical cord, attaching a third cannula to a second artery of the umbilical cord, and connecting one or more of the first, second and third cannulae to the oxygenator via an oxygenation circuit comprising tubing, wherein at least one of the cannulae is in fluid connection with the fetus' circulatory system and the umbilical cord is connected to the oxygenator such that deoxygenated blood is delivered from the fetus to the oxygenator and oxygenated blood is delivered from the oxygenator to the fetus via the tubing; and (b) extracorporeally irradiating at least a portion of the fetus' blood through with actinic radiation at a wavelength of from about 430 nm to about 490 nm via a photo-therapy device. As used herein, the term “treating” includes lowering serum bilirubin levels and halting the increase of serum bilirubin levels. In one embodiment, the fetus' blood is exposed with actinic radiation through at least a portion of the tubing, the oxygenator, or both.

Phototherapy devices are preferably powered by low voltage battery or by a low voltage power source. As mentioned above, the preferred wavelength for photo-therapy treatment of elevated bilirubin levels is in the range of from about 430 nm to about 490 nm. In some embodiments, it is preferable to use wavelengths in the range of from about 460 nm to about 490 nm light emitting diodes (“LED”) for treating a premature fetus by exhibiting one or more of the following attributes: a narrow emission wavelength, focused angle of radiation, or a very high intensity. An exemplary LED photo therapy source can be obtained from USHIO OPTO SEMICONDUCTOR, INC., available at https://www.ushio.com/leds/#led-lighting. Yet another exemplary phototherapy light source is a light diode.

The device disclosed herein allows for targeting blood within the extracorporeal circuit's flexible plastic tubing or transparent or translucent oxygenator in the blood circuit to achieve the desired biological effect. The tubing may be transparent, translucent or it may have only a transparent or translucent portion (i.e., a window) through which the fetal blood can be irradiated. The tubing may be irradiated from one side or from more than one side (e.g., 360°).

A typical flexible plastic tubing for such applications has an outside diameter (OD) of 5/16″ and a 1/16″ wall thickness. In an embodiment, approximately a 125 mm length of such tubing is illuminated.

In one embodiment, the photo-therapy device is a stationary blue lamp or a fluorescent tube. In another embodiment, the photo-therapy device is a hand-held LED light strip. In further embodiments, the photo-therapy device can be a LED gun, filament, or lamp. In yet another embodiment, the photo-therapy device can be one or more LEDs.

In another embodiment, the photo-therapy device is a device that attaches directly (e.g., clips onto) to the flexible plastic tubing when needed for non-invasive blood phototherapy to treat the blood at a certain serum level to decrease the serum bilirubin levels. Detection of such levels is obtained using point of care (POC) analyzers know to those skilled in the art.

The phototherapy device resides within the interior volume 4134 of system 10. Within interior volume 4124, there are one or more interior subsections defined by opaque wall(s), curtains, or the like, that separates a portion of the oxygenation circuit (not shown) from interior volume 4132 wherein chamber 100 and the premature fetus 2 resides. The opaque wall(s) minimize the light from the phototherapy device entering into chamber 100 and exposing the premature fetus 2 directly or indirectly with the light.

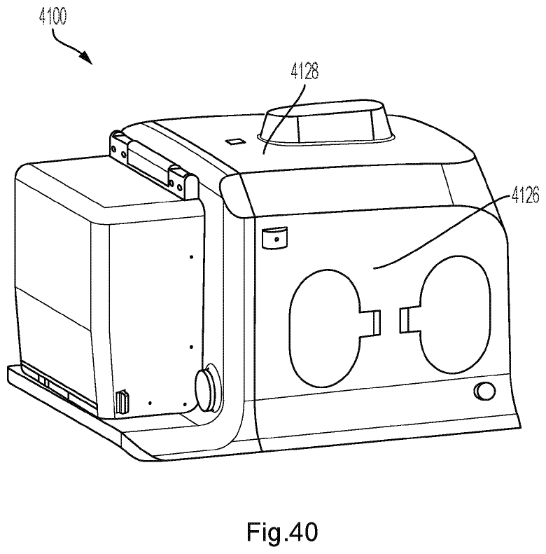

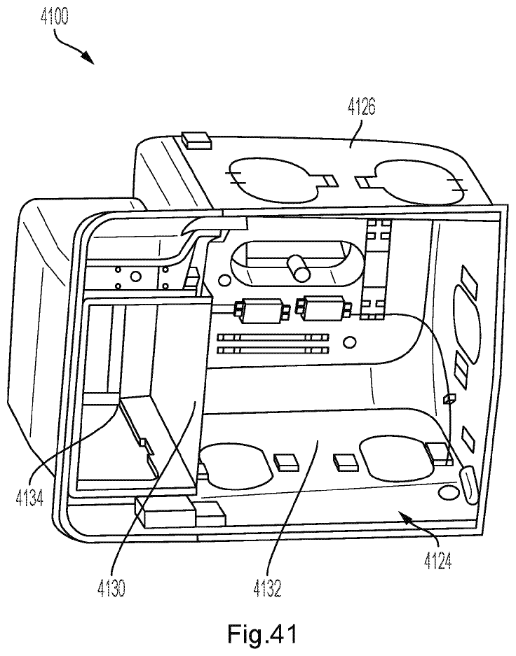

Referring to FIGS. 40 and 41, the system can include a canopy 4100. The canopy 4100 can include side walls 4126 and a lid 4128. The sidewalls 4126 and lid 4128 can be opaque. The sidewalls 4126 and lid 4128 can be a monolithic construct. The sidewalls 4126 and lid 4128 can define an interior space 4124 configured to receive the chamber 100. The sidewalls 4126 and lid 4128 can limit the amount of light that reaches the interior space 4124 to about 1.2 lux or below.

The canopy 4100 can include a divider 4130 that divides the interior space 4124 into a first portion 4132 and a second portion 4134. The chamber 100 can be received in the first portion 4132. The phototherapy device 3500 (described below) can be received in the second portion 4134. The divider 4130 can be opaque. The divider 4130 can limit the amount of light that reaches the first portion 4132 of the interior space 4124 to about 1.2 lux or below. The canopy 4100 can be used in place of the housing 20. The housing 20 of FIG. 1 can also include the divider 4130.

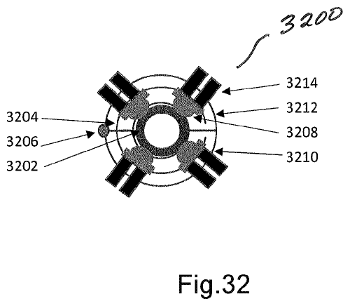

Referring to FIG. 32, an exemplary embodiment of a photo-therapy device 3200 suitable for use to illuminate fetal blood flowing through the inside of a flexible plastic tube 3202. Device 3200 has a “clam shell” design with the lower half 3210 and upper half 3212 being movably attached via hinge 3206. Photo-therapy device 3200 includes LED lights 3208 on a flex circuit 3204. Heat sinks 3214 are attached to the LEDs for dissipating heat away from the flexible plastic tube 3202.

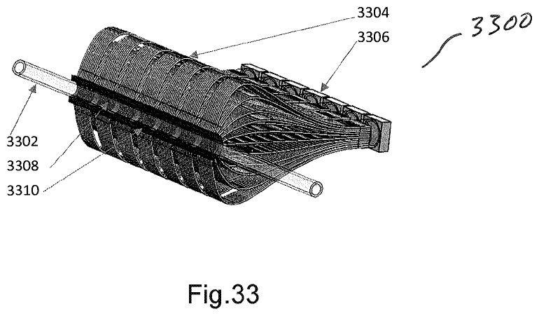

FIG. 33 illustrates another exemplary embodiment of a phototherapy device. Here, photo-therapy device 3300 is also a clam-shell design wherein shell 3310 has an upper and a lower portion that matingly fit around flexible tubing 3302. In the embodiment shown, each half “shell” comprises a separate unit containing light source(s) 3306, such as LEDs, with the light conveyed to the clam shell structure by optical fibers or light pipes 3304. Large core multi-mode fibers with large numerical aperture are used to capture the maximum amount of light output from the source 3306 in the fiber. Within the tubing interface (clam shell) the fibers could be positioned radially to expose the tubing to the maximum irradiance flux density. In this embodiment, the fiber ends are potted in a medium to hold the ends essentially perpendicular to the target tubing. The potting material could constitute the shell, or it could be secured within an outer hinged or flexible structure. The fibers can be separated into bundles to be addressed by the light sources (LEDs) in the most efficient form. The fiber bundles can comprise individual separate fibers or fused groups of fibers.

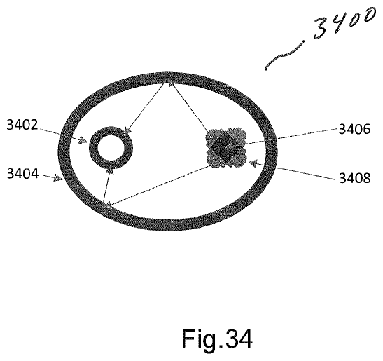

FIG. 34 illustrates yet another embodiment of a photo-therapy device for use in the method disclosed herein. The device 3400 is in the shape of an elongated elliptical reflective cavity 3404 with the tubing 3402 at one focus and the light sources 3406 at the other. The light source can be LEDs mounted on a heat sink 3408 or a fluorescent tube. The light is reflected from the source focus to the tubing at the opposite focus. The light source and the tubing are separated minimizing the heat transfer to the tubing. One or more heat sinks 3408 may be employed to prevent the temperature of the fetus' blood from rising more than about 2.0° F. or less, about 1.5° F. or less, or about 1.0° F. or less.

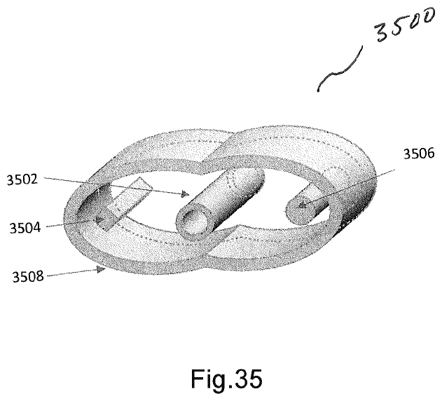

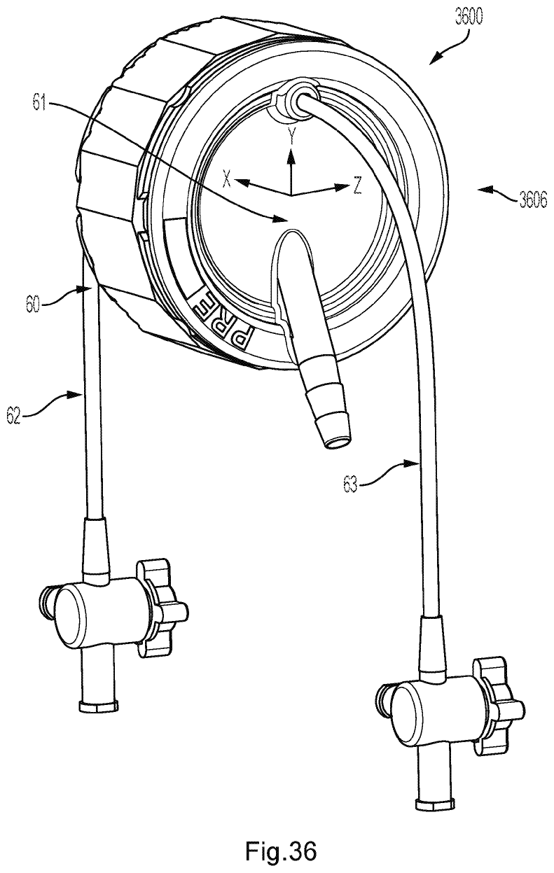

FIG. 35 illustrates still yet another embodiment of a photo-therapy device 3500 for use in the method disclosed herein. In this embodiment, an elongated double elliptical cavity has a reflective shell 3508 with the tubing 3502 located at the common focus would produce additional illumination as the light source 3506 is doubled and impact of the sources blocking reflected light is mitigated. Light sources 3506 are located at the noncommon foci in the form of LEDs or fluorescent tubes. The ends of the ellipses are covered to block the light from escaping.

The oxygenator is yet another source for irradiating blood from the fetus. FIG. 36 illustrates an embodiment wherein the oxygenator is exposed to the phototherapy device on at least one exterior surface of the oxygenator. In the embodiment shown in FIG. 36, at least one of the face plates of oxygenator 60 or face plate 61, is exposed to the phototherapy device. At least a portion of face plate 61 is transparent to allow the blood contained within oxygenator 60 to be exposed to a light source 3606 such as a UV light gun emitting from photo-therapy device 3600. In this or other embodiments at least one of the transparent face plates of the oxygenator is dimpled, angled, refracted, or has some other surface aspect(s) or feature(s) that maximizes the surface area of blood exposed to light source 3606 and/or diffuse the light from the light source within the oxygenator. At least a portion of face plate 61 is transparent to allow the blood contained within oxygenator 60 to be exposed to a light source 3606 such as a UV light gun emitting from photo-therapy device 3600. The face plate 61 can be shaped so as to bend the light moving through the face plate 61. For example, the face plate 61 can be shaped to diffuse the light moving through the face plate 61. In another example, the face plate 61 can be shaped to focus the light moving through the face plate 61. The face plate 61 can have a convex or concave shape so as to bend the light moving through the face plate 61. In another embodiment, photo-therapy device, such as a LED assembly, for example, can clip over the oxygenator 60 such that LED light sources are placed in the well area of one or both of the face plates. The oxygenator 60 has two sides with significant surface area. Referring to FIG. 36, line 62 represents the flow of blood exiting oxygenator 60 or “post” and line 63 represents the flow of blood entering oxygenator 60 or “pre”. The patient's blood spreads out inside of the oxygenator across the blood face plates (transparent or translucent) entering and exiting the oxygenator. The phototherapy device is placed proximal to at least one or both sides of oxygenator 60. Having a light source on at least one or both sides of oxygenator 61 makes this produce potentially more blood surface area then the tubing approach. Besides the phototherapy device comprising an IR gun or 3606, other embodiments can be a clam shell or ear muff design that clamps or mounts onto the oxygenator and exposes at least a portion of the transparent surfaces to the light source contained therein.

In certain embodiments, phototherapy device is a plate containing one or more LED lights arranged in an area that concentrates the light source to the face plate(s) of the oxygenator. In yet another embodiment, the phototherapy device is a ring such as a circle, oval, or elliptical ring, or LED or other light sources attached to or proximal to one or more of the transparent surface(s) of the oxygenator. In these or other embodiments, an infrared filter may be placed between the light source and at least one of the oxygenator's transparent surface(s) to reflect or focus the light on the surface(s). In these or other embodiments, a heat sink(s) or other means can be used to lessen the amount of heat dissipation.

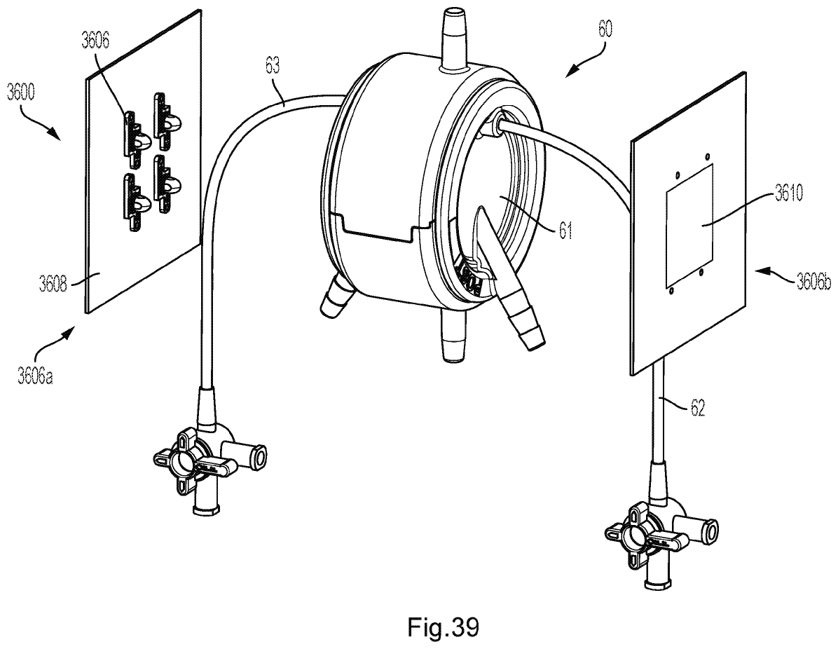

Referring to FIG. 39, the photo-therapy device 3600 can include a light source 3606 such as an LED array. The LED array can include one or more LEDs configured to emit UV light. In some examples, the LED wavelength is adjustable. The LED array can include a first LED and a second LED. The first LED can be configured to emit light at a first wavelength and the second LED can be configured to emit light at a second wavelength. In some examples, the first and second wavelength are the same. In other examples, the first and second wavelengths are different. The LED array can include 470 nm LED lights arranged in a grid the substrate 3608. Each LED light can be separated by a distance of about 10 cm, about 7 cm, about 5 cm, or about 2 cm relative to each other.

The substrate 3608 can be configured to transfer heat generated by the light source 3606 away from the oxygenator 60. The light source 3606 can be coupled to a substrate 3608. The substrate 3608 can be manufactured from a material having high thermal conductivity. For example, the substrate 3608 can be manufactured from metal (e.g., aluminum, brass, steel, bronze, copper, or iron). A heat sink 3610 can be thermally coupled to the substrate 3608 to dissipate heat from the light source 3606. The heat sink 3610 can be fixed to the substrate 3608. The heat sink 3610 can include a plurality of fins. The heat sink 3610 can be a passive heat sink. The heat sink 3610 can be an active heat sink that includes a fan or blower. The heat sink 3610 can be selected based on the heat output of the light source 3606 and the distance of the light source 3606 from the oxygenator 60.

The photo-therapy device 3600 can include two light sources 3606. The first light source 3606a and second light source 3606b can be the same. For example, each of the first and second light sources 3606a and 3606b can include an LED array. The first and second light sources 3606a, 3606b can include the same number of LEDs in the LED array. The first and second light sources 3606a, 3606b can each be configured to emit light at the same wavelength. The first and second light sources 3606a, 3606b can be positioned equidistant from the oxygenator 60. The first light source 3606a may be positioned closer to the oxygenator when the first light source 3606a is on the blood entrance side of the oxygenator 60.

In another embodiment, the method for extracorporeally treating hyperbilirubinemia in a premature fetus' blood in an ex-utero environment is independent of the specific ex-utero apparatus/system disclosed above. In this embodiment, any premature fetus connected to any ex-utero environment comprising an extracorporeal circuit can be treated for hyperbilirubinemia by exposing the fetus' blood through flexible tubing through which the fetus' blood is circulating. Thus, in an embodiment, the method for extracorporeally treating hyperbilirubinemia in a premature fetus' blood in an ex-utero environment comprises connecting a premature fetus to an ex-utero system configured to provide oxygen to the fetus via a blood circulation system comprising tubing through which the fetus' blood flows; and extracorporeally irradiating at least a portion of the fetus' blood through at least a portion of the tubing with actinic radiation at a wavelength of from about 430 nm to about 490 nm via a photo-therapy device.

### EXAMPLES

Previous experiments conducted with phototherapy devices having a ring LED configuration such as the embodiment shown in FIG. 32 have been used on a 25 centimeter (cm) segment of transparent flexible tubing similar to that used in the extracorporeal system. The results were mixed. While there may have been a decrease in serum bilirubin levels in the blood sample, the heat generation of the phototherapy device was undesirable. A clinical target of about 2 to about 4 milligrams/deciliter (mg/dL) per day total bilirubin (tBili) reduction can be achieved with ring LED phototherapy device. However, the rise in blood temperature was not ideal (<0.5° C. target). Further the flexible tubing had limited availability of blood surface area for exposure. While the blood in the tubing exposed to the phototherapy light was treated, the blood in the center and opposite side of the tubing was not treated to the same extent.

An experimental setup like the embodiment shown in FIG. 39 was tested to see its effect on the bilirubin serum level in porcine blood. In the examples, the bilirubin serum level was measuring employing a bilirubin oxidase colorimetric reaction and using the hepatic function panel carriage of the Abbott Point of Care Piccolo analyzer. Exposure of the light source to one or more of the oxygenator faceplates provides a larger blood surface area than the ring LED configuration. The phototherapy device consisted of an array of four 470 nm LED lights arranged in a grid on a 4 centimeter aluminum plate with each LED light separated by a distance of 2 cm relative to each other. The aluminum plate was placed at a distance of 6.5 cm from the face plate of the oxygenator wherein at least a portion of its faceplate is transparent. Two phototherapy devices were used to compare the effects on bilirubin serum level when exposed to two faces of the oxygenator (e.g., pre-oxygenator and post-oxygenator blood) at 100% irradiance, two phototherapy devices at 50% irradiance; and one phototherapy device at a 100% irradiance exposed to the pre-oxygenator side. The porcine blood temperature prior to treatment was approximately 39° C. The initial bilirubin level (tBili) was 6.4 mg/dL. The flow rate of blood through the oxygenator was about 50 ml/min and the sweep gas (95% air and 5% CO₂) was 100 milliliter/minute (ml/min). The study was conducted over a 24 hour period.

The following TABLE I shows the results of exposure to irradiance levels of 50% and 100% radiance on total and direct bilirubin using the configuration described above on exposure of the phototherapy device to two face plates of an oxygenator at 50% and 100% radiance and one face plate of the oxygenator or the pre-oxygenator side at 100% radiance:

TABLE I

| Irradiance | Total Bilirubin | Direct Bilirubin |
|---|---|---|
| (%) | (mg/dL/day) | (mg/mL/day) |
| 100 | −4.4 | 2.54 |
| 50 | −2.2 | 1.26 |
| Pre-plate 100 | −2.01 | 1.03 |

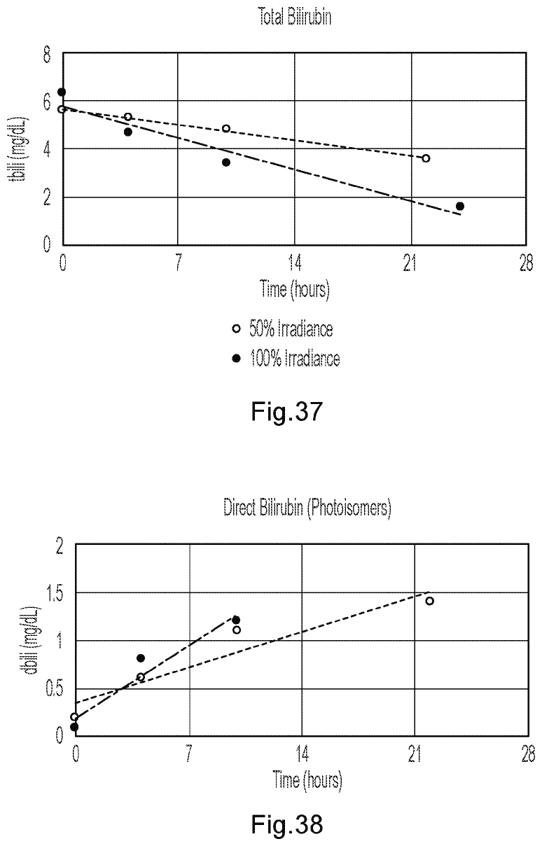

FIG. 37 and FIG. 38 show the relation between total and direct bilirubin over time (hours), respectively for different arrangements of the phototherapy device and irradiance levels. The following TABLE II provides the results of exposure to irradiance levels of 50% and 100% radiance wherein the phototherapy device is the LED diode array on an aluminum plate applied to the pre-plate oxygenator face and post-plate oxygenator at different radiance levels and pre-plate oxygenator face at 100% relative to temperature. The surface temperature of the oxygenator was measured using an infrared (“IR”) gun. There was minimal change in the temperature as shown by the delta in TABLE II:

TABLE II

| Irradiance | Lights | Pre-plate temp | Post-plate temp |
|---|---|---|---|
| (%) | (ON/OFF) | (° C.) | (° C.) |
| 100 | ON | 39.8 | 38.0 |
| | OFF | 38.8 | 38.1 |
| | Δ | 1.0 | 0.1 |
| 50 | ON | 39.7 | 39.0 |
| | OFF | 38.7 | 38.2 |
| | Δ | 1.0 | 0.8 |
| Pre-plate 100 | ON | 40.8 | 40.4 |
| | OFF | 40.6 | 40.4 |
| | Δ | 0.2 | 0 |

A comparison of a ring embodiment photo therapy device such as the embodiment in FIG. 32 and an oxygenator face plate phototherapy device such as the embodiment in FIG. 39 is provided in TABLE III.

TABLE III

| | | | Initial | | | | A Blood | Surface |
|---|---|---|---|---|---|---|---|---|
| | No. | Irradiance | Bilirubin | tBili rate | dBili rate | Anamolies | temp. rise | temp. |
| Design | LEDs | (%) | (mg/dL) | (mg/dL/day) | (mg/dL/day) | at light area | (° C.) | (° C.) |
| Ring | 4 | 100 | 7.3 | −3.51 | 3.60 | Non- | Post | 55.6 |
| (470 nm) | | | | | | biologica1 | −2.54 | |
| | | | | | | coating | (8 lights) | |
| Ring | 4 | 75 | 4.2 | −2.45 | N/A | RBC | Pre | 43.2 |
| | | | | | | coagulation | −1.62; | |
| | | | | | | | Post | |
| | | | | | | | −1.64 | |
| Ring | 4 | 50 | 7.7 | −1.37 | 1.97 | N/A | Pre | 40.1 |
| | | | | | | | −0.35; | |
| | | | | | | | Post | |
| | | | | | | | −0.56 | |
| Oxygenat | 8 | 100 | 6.3 | −4.4 | 2.54 | N/A | N/A | Pre |
| or Face | | | | | | | | −39.8; |
| Plates | | | | | | | | Post |
| | | | | | | | | −38.0 |
| Oxygenat | 8 | 50 | 5.6 | −2.2 | 1.26 | N/A | Pre | Pre |
| or Face | | | | | | | −0.30; | −39.7; |
| Plates | | | | | | | Post | Post |
| | | | | | | | −0.25 | −39.0 |

TABLE IV provides the irradiance comparison between the phototherapy device comprising a ring such as the embodiment in FIG. 32 and the oxygenator face plate(s) such as the embodiment shown in FIG. 39:

TABLE IV

| | | | | | | Spectral |
|---|---|---|---|---|---|---|
| | | Average | | | Total | flux |
| | Area per | Irradiance | Spectral | | Area | delivered to |
| Design | LED (cm²) | (uw/cm²) | flux (uw) | No. LEDs | (cm²) | blood (mw) |
| Ring | 0.81 | 4000 (through | 3238 | 4 | 3.24 | 12.96 |
| | | tube wall); 1 | | | | |
| | | LED | | | | |
| Oxygenator Face | 4.0 | ~230 at 6.5 cm | 1840 | 2 × 4 | 29.0 | 53.36 |
| Plates (@ 100%) | | (9000 at LED | | | | |
| | | surface) | | | | |
| Oxygenator Face | 4.0 | ~230 at 6.5 cm | 920 | 2 × 4 | 29.0 | 26.68 |
| Plates (@ 50%) | | (9000 at LED | | | | |
| | | surface) | | | | |
| Oxygenator Face | 4.0 | ~230 at 6.5 cm | 920 | 1 × 4 | 14.5 | 26.68 |
| Plate (@ 100% on | | (9000 at LED | | | | |
| Pre-Oxygenator | | surface) | | | | |
| side) | | | | | | |

It will be appreciated that the foregoing description provides examples of the disclosed system and methods. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range including the stated ends of the range, unless otherwise indicated herein, and each separate value is incorporated into the specification as if it were individually recited herein. All methods described herein can be performed in any suitable order unless otherwise indicated herein or otherwise clearly contradicted by context. The term “about” as used herein in reference to numerical ranges can mean the stated value+/−1%, 2%, 3%, 4%, 5% or 10%.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A method for treating hyperbilirubinemia in a premature fetus' blood in an ex-utero environment, the method comprising the steps of:
a. connecting a premature fetus to an ex-utero system configured to provide oxygen to the fetus, wherein the connecting step comprises the steps of attaching a first cannula to a vein of the umbilical cord, attaching a second cannula to a first artery of the umbilical cord, attaching a third cannula to a second artery of the umbilical cord, and connecting one or more of the first, second and third cannulae to the oxygenator via an oxygenation circuit comprising tubing, wherein at least one of the cannulae is in fluid connection with the fetus' circulatory system and the umbilical cord is connected to the oxygenator such that deoxygenated blood is delivered from the fetus to the oxygenator and oxygenated blood is delivered from the oxygenator to the fetus via the tubing; and
b. extracorporeally irradiating at least a portion of the fetus' blood through at least a portion of the tubing, the oxygenator, or both with actinic radiation at a wavelength of from about 430 nm to about 490 nm via a photo-therapy device.

2. The method of claim 1 wherein the wavelength of the actinic radiation is from about 460 nm to about 490 nm.

3. The method of claim 2 wherein the wavelength of the actinic radiation is from about 470 nm to about 490 nm.

4. The method of claim 1 wherein the photo-therapy device is a clip-on device.

5. The method of claim 1 wherein the photo-therapy device is a blue lamp or fluorescent tube.

6. The method of claim 1 wherein the photo-therapy device comprises at least one LED capable of illumination at a wavelength of from about 460 nm to about 490 nm, wherein the at least one LED is located in a clam shell-type housing, wherein the housing comprises a hinge.

7. A method for extracorporeally treating hyperbilirubinemia in a premature fetus' blood in an ex-utero environment comprises connecting a premature fetus to an ex-utero system configured to provide oxygen to the fetus via a blood circulation system comprising tubing through which the fetus' blood flows; and extracorporeally irradiating at least a portion of the fetus' blood through at least a portion of the tubing with actinic radiation at a wavelength of from about 430 nm to about 490 nm via a photo-therapy device.

8. The method of claim 7 wherein the wavelength of the actinic radiation is from about 460 nm to about 490 nm.

9. The method of claim 7 wherein the photo-therapy device is a clip-on device.

10. The method of claim 7 wherein the photo-therapy device is a blue lamp or fluorescent tube.
